SEQUENCE LISTING (148 sequences)

SEQ ID NO: 1 - DNA, 52 bp, Homo sapiens
gttttccttgacaatctactgtgccactgtgctactaactcttgtttacaaa

SEQ ID NO: 2 - RNA, 22 nt, Homo sapiens
uguaaacauccucgacuggaag

SEQ ID NO: 3 - RNA, 22 nt, Homo sapiens
cuuucagucggauguuugcagc

SEQ ID NO: 4 - RNA, 22 nt, Homo sapiens
uguaaacauccuacacucagcu

SEQ ID NO: 5 - RNA, 22 nt, Homo sapiens
cugggagguggauguuuacuuc

SEQ ID NO: 6 - RNA, 23 nt, Homo sapiens
uguaaacauccuacacucucagc

SEQ ID NO: 7 - RNA, 22 nt, Homo sapiens
cugggagaggguuguuuacucc

SEQ ID NO: 8 - RNA, 22 nt, Homo sapiens
cugggagaaggcuguuuacucu

SEQ ID NO: 9 - RNA, 22 nt, Homo sapiens
uguaaacauccccgacuggaag

SEQ ID NO: 10 - RNA, 22 nt, Homo sapiens
cuuucagucagauguuugcugc

SEQ ID NO: 11 - RNA, 22 nt, Homo sapiens
uguaaacauccuugacuggaag

SEQ ID NO: 12 - RNA, 22 nt, Homo sapiens
cuuucagucggauguuuacagc

SEQ ID NO: 13 - RNA, 21 nt, Homo sapiens
ugauuguccaaacgcaauucu

SEQ ID NO: 14 - RNA, 22 nt, Homo sapiens
agaguugagucuggacgucccg

SEQ ID NO: 15 - RNA, 22 nt, Homo sapiens
agaauuguggcuggacaucugu

SEQ ID NO: 16 - RNA, 22 nt, Homo sapiens
agauguccagccacaauucucg

SEQ ID NO: 17 - RNA, 23 nt, Homo sapiens
agaauugcguuuggacaaucagu

SEQ ID NO: 18 - RNA, 23 nt, Homo sapiens
uacuccagagggcgucacucaug

SEQ ID NO: 19 - RNA, 23 nt, Homo sapiens
ugauuguagccuuuuggaguaga

SEQ ID NO: 20 - RNA, 23 nt, Homo sapiens
cggggccguagcacugucugaga

SEQ ID NO: 21 - RNA, 23 nt, Homo sapiens
gggggccgauacacuguacgaga

SEQ ID NO: 22 - RNA, 21 nt, Homo sapiens
ucacagugaaccggucucuuu

SEQ ID NO: 23 - RNA, 22 nt, Homo sapiens
uagcuuaucagacugauguuga

SEQ ID NO: 24 - RNA, 21 nt, Homo sapiens
caacaccagucgaugggcugu

SEQ ID NO: 25 - RNA, 22 nt, Homo sapiens
agccccugcccaccgcacacug

SEQ ID NO: 26 - RNA, 22 nt, Homo sapiens
cugugcgugugacagcggcuga

SEQ ID NO: 27 - RNA, 22 nt, Homo sapiens
gggguuccuggggaugggauuu

SEQ ID NO: 28 - RNA, 21 nt, Homo sapiens
aucacauugccagggauuucc

SEQ ID NO: 29 - RNA, 22 nt, Homo sapiens
uggguuccuggcaugcugauuu

SEQ ID NO: 30 - RNA, 21 nt, Homo sapiens
aucacauugccagggauuacc

SEQ ID NO: 31 - RNA, 22 nt, Homo sapiens
aucacauugccagugauuaccc

SEQ ID NO: 32 - RNA, 22 nt, Homo sapiens
ggugcagugcugcaucucuggu

SEQ ID NO: 33 - RNA, 21 nt, Homo sapiens
ugagaugaagcacuguagcuc

SEQ ID NO: 34 - RNA, 18 nt, Homo sapiens
ugagaugacacuguagcu

SEQ ID NO: 35 - RNA, 23 nt, Homo sapiens
uuuggcacuagcacauuuuugcu

SEQ ID NO: 36 - RNA, 22 nt, Homo sapiens
aaucaugugcagugccaauaug

SEQ ID NO: 37 - RNA, 21 nt, Homo sapiens
uuuugcaccuuuuggagugaa

SEQ ID NO: 38 - RNA, 22 nt, Homo sapiens
cuuggcaccuagcaagcacuca

SEQ ID NO: 39 - RNA, 22 nt, Homo sapiens
agugccugcuaugugccaggca

SEQ ID NO: 40 - RNA, 22 nt, Homo sapiens
aaaguucugagacacuccgacu

SEQ ID NO: 41 - RNA, 22 nt, Homo sapiens
ucagugcacuacagaacuuugu

SEQ ID NO: 42 - RNA, 22 nt, Homo sapiens
aaguucuguuauacacucaggc

SEQ ID NO: 43 - RNA, 21 nt, Homo sapiens
ucagugcaugacagaacuugg

SEQ ID NO: 44 - RNA, 24 nt, Homo sapiens
uuuggcaaugguagaacucacacu

SEQ ID NO: 45 - RNA, 21 nt, Homo sapiens
ugguucuagacuugccaacua

SEQ ID NO: 46 - RNA, 22 nt, Homo sapiens
agggcuuagcugcuugugagca

SEQ ID NO: 47 - RNA, 21 nt, Homo sapiens
uucacaguggcuaaguuccgc

SEQ ID NO: 48 - RNA, 22 nt, Homo sapiens
agagcuuagcugauuggugaac

SEQ ID NO: 49 - RNA, 21 nt, Homo sapiens
uucacaguggcuaaguucugc

SEQ ID NO: 50 - RNA, 22 nt, Homo sapiens
caguuaucacagugcugaugcu

SEQ ID NO: 51 - RNA, 21 nt, Homo sapiens
uacaguacugugauaacugaa

SEQ ID NO: 52 - RNA, 21 nt, Homo sapiens
uggaggagaaggaaggugaug

SEQ ID NO: 53 - RNA, 22 nt, Homo sapiens
ugagaaccacgucugcucugag

SEQ ID NO: 54 - RNA, 24 nt, Homo sapiens
ucagaacaaaugccgguucccaga

SEQ ID NO: 55 - RNA, 22 nt, Homo sapiens
uucuggaauucugugugaggga

SEQ ID NO: 56 - RNA, 21 nt, Homo sapiens
gaagugugccguggugugucu

SEQ ID NO: 57 - RNA, 22 nt, Homo sapiens
gcucugacuuuauugcacuacu

SEQ ID NO: 58 - RNA, 23 nt, Homo sapiens
cagugcaauaguauugucaaagc

SEQ ID NO: 59 - RNA, 23 nt, Homo sapiens
cagugcaaugauauugucaaagc

SEQ ID NO: 60 - RNA, 22 nt, Homo sapiens
aaaaguaauugcgaguuuuacc

SEQ ID NO: 61 - RNA, 22 nt, Homo sapiens
caaaacuggcaauuacuuuugc

SEQ ID NO: 62 - RNA, 22 nt, Homo sapiens
aaaaguaauugugguuuuggcc

SEQ ID NO: 63 - RNA, 22 nt, Homo sapiens
caagaaccucaguugcuuuugu

SEQ ID NO: 64 - RNA, 22 nt, Homo sapiens
aaaaguaauugcgguuuuugcc

SEQ ID NO: 65 - RNA, 22 nt, Homo sapiens
caaaaaucucaauuacuuuugc

SEQ ID NO: 66 - RNA, 22 nt, Homo sapiens
aaaaguaauugugguuuuugcc

SEQ ID NO: 67 - RNA, 22 nt, Homo sapiens
caaaaaccacaguuucuuuugc

SEQ ID NO: 68 - RNA, 22 nt, Homo sapiens
aaaaacugagacuacuuuugca

SEQ ID NO: 69 - RNA, 19 nt, Homo sapiens
aaaaacuguaauuacuuuu

SEQ ID NO: 70 - RNA, 23 nt, Homo sapiens
ugcaaaaguaauugcaguuuuug

SEQ ID NO: 71 - RNA, 22 nt, Homo sapiens
aaaacuguaauuacuuuuguac

SEQ ID NO: 72 - RNA, 22 nt, Homo sapiens
aaaaguaaucgcgguuuuuguc

SEQ ID NO: 73 - RNA, 23 nt, Homo sapiens
caaaaaccgcaauuacuuuugca

SEQ ID NO: 74 - RNA, 22 nt, Homo sapiens
aaaaguaauugcggauuuugcc

SEQ ID NO: 75 - RNA, 22 nt, Homo sapiens
aaaaguaauugcggucuuuggu

SEQ ID NO: 76 - RNA, 22 nt, Homo sapiens
aaaaguacuugcggauuuugcu

SEQ ID NO: 77 - RNA, 22 nt, Homo sapiens
aaaaguauuugcggguuuuguc

SEQ ID NO: 78 - RNA, 21 nt, Homo sapiens
caaagguauuugugguuuuug

SEQ ID NO: 79 - RNA, 22 nt, Homo sapiens
caaaaguaauuguggauuuugu

SEQ ID NO: 80 - RNA, 22 nt, Homo sapiens
aaaaguaauugcgguuuuugcc

SEQ ID NO: 81 - RNA, 22 nt, Homo sapiens
ccaaaacugcaguuacuuuugc

SEQ ID NO: 82 - RNA, 22 nt, Homo sapiens
uagcaaaaacugcaguuacuuu

SEQ ID NO: 83 - RNA, 22 nt, Homo sapiens
gcuggugcaaaaguaauggcgg

SEQ ID NO: 84 - RNA, 23 nt, Homo sapiens
auggccaaaacugcaguuauuuu

SEQ ID NO: 85 - RNA, 21 nt, Homo sapiens
caaaagugaucgugguuuuug

SEQ ID NO: 86 - RNA, 25 nt, Homo sapiens
aaaaaccacaauuacuuuugcacca

SEQ ID NO: 87 - RNA, 23 nt, Homo sapiens
caaagacugcaauuacuuuugcg

SEQ ID NO: 88 - RNA, 22 nt, Homo sapiens
agcuacaguuacuuuugcacca

SEQ ID NO: 89 - RNA, 23 nt, Homo sapiens
aaaaguaacugcgguuuuugccu

SEQ ID NO: 90 - RNA, 23 nt, Homo sapiens
ugcaaaaguaauugcaguuuuug

SEQ ID NO: 91 - RNA, 20 nt, Homo sapiens
uaaaaacugcaauuacuuuc

SEQ ID NO: 92 - RNA, 22 nt, Homo sapiens
aaaaguaaucacuguuuuugcc

SEQ ID NO: 93 - RNA, 23 nt, Homo sapiens
caaaaaccgcaauuacuuuugca

SEQ ID NO: 94 - RNA, 25 nt, Homo sapiens
aaaaaccacaauuacuuuugcacca

SEQ ID NO: 95 - RNA, 22 nt, Homo sapiens
aaaaguaauuguggauuuugcu

SEQ ID NO: 96 - RNA, 22 nt, Homo sapiens
caaaaaccggcaauuacuuuug

SEQ ID NO: 97 - RNA, 22 nt, Homo sapiens
gaaaacgacaaugacuuuugca

SEQ ID NO: 98 - RNA, 21 nt, Homo sapiens
caaaaacugcaauuacuuuca

SEQ ID NO: 99 - RNA, 21 nt, Homo sapiens
aaagguaauugugguuucugc

SEQ ID NO: 100 - RNA, 20 nt, Homo sapiens
aaaagugauugcaguguuug

SEQ ID NO: 101 - RNA, 22 nt, Homo sapiens
caaaaacugcaguuacuuuugc

SEQ ID NO: 102 - RNA, 22 nt, Homo sapiens
aaagguaauugcaguuuuuccc

SEQ ID NO: 103 - RNA, 23 nt, Homo sapiens
ugcaaaaguaauugcaguuuuug

SEQ ID NO: 104 - RNA, 21 nt, Homo sapiens
uaaaaacugcaauuacuuuua

SEQ ID NO: 105 - RNA, 21 nt, Homo sapiens
aaaaguaacugcgguuuuuga

SEQ ID NO: 106 - RNA, 22 nt, Homo sapiens
aacggcaaugacuuuuguacca

SEQ ID NO: 107 - RNA, 22 nt, Homo sapiens
aaaaguaauugcgguuuuugcc

SEQ ID NO: 108 - RNA, 22 nt, Homo sapiens
caaaaacugcaguuacuuuugu

SEQ ID NO: 109 - RNA, 20 nt, Homo sapiens
aaaaggcauugugguuuuug

SEQ ID NO: 110 - RNA, 22 nt, Homo sapiens
agaaguaacuacgguuuuugca

SEQ ID NO: 111 - RNA, 22 nt, Homo sapiens
aaagaccgugacuacuuuugca

SEQ ID NO: 112 - RNA, 19 nt, Homo sapiens
aaaaguaauugcggucuuu

SEQ ID NO: 113 - RNA, 19 nt, Homo sapiens
aaaaaccacaauuacuuuu

SEQ ID NO: 114 - RNA, 22 nt, Homo sapiens
gaaaguaauugcuguuuuugcc

SEQ ID NO: 115 - RNA, 22 nt, Homo sapiens
caaaaacugcaauuacuuuugc

SEQ ID NO: 116 - RNA, 21 nt, Homo sapiens
aaaaguaauugcaguuuuugc

SEQ ID NO: 117 - RNA, 21 nt, Homo sapiens
uaaaacugcaguuauuuuugc

SEQ ID NO: 118 - RNA, 22 nt, Homo sapiens
aaaaguaauugcggguuuugcc

SEQ ID NO: 119 - RNA, 22 nt, Homo sapiens
uaaaacccacaauuauguuugu

SEQ ID NO: 120 - RNA, 22 nt, Homo sapiens
aaaaguuauugcgguuuuggcu

SEQ ID NO: 121 - RNA, 21 nt, Homo sapiens
caaaaccgcaguaacuuuugu

SEQ ID NO: 122 - RNA, 21 nt, Homo sapiens
aaaaguaauugcgguuuuugc

SEQ ID NO: 123 - RNA, 21 nt, Homo sapiens
uggcaguuacuuuugcaccag

SEQ ID NO: 124 - RNA, 18 nt, Homo sapiens
aaaaguacuugcggauuu

SEQ ID NO: 125 - RNA, 20 nt, Homo sapiens
aaaacugcaguuacuuuugc

SEQ ID NO: 126 - RNA, 20 nt, Homo sapiens
gugcaaaagucaucacgguu

SEQ ID NO: 127 - RNA, 22 nt, Homo sapiens
agaaguaauugcgguuuugcca

SEQ ID NO: 128 - RNA, 21 nt, Homo sapiens
aaaaguaauugugguuuuugc

SEQ ID NO: 129 - RNA, 22 nt, Homo sapiens
caaaaccgcgauuacucuugca

SEQ ID NO: 130 - RNA, 22 nt, Homo sapiens
caaaagugauugugguuuuugc

SEQ ID NO: 131 - RNA, 21 nt, Homo sapiens
aaaaacugcaaucacuuuugc

SEQ ID NO: 132 - RNA, 19 nt, Homo sapiens
auggauaaggcuuuggcuu

SEQ ID NO: 133 - RNA, 21 nt, Homo sapiens
cugacuguugccguccuccag

SEQ ID NO: 134 - RNA, 23 nt, Homo sapiens
acuugggcacugaaacaaugucc

SEQ ID NO: 135 - RNA, 21 nt, Homo sapiens
uacugcagacaguggcaauca

SEQ ID NO: 136 - RNA, 22 nt, Homo sapiens
uacugcagacguggcaaucaug

SEQ ID NO: 137 - RNA, 22 nt, Homo sapiens
ugauugguacgucuguggguag

SEQ ID NO: 138 - RNA, 20 nt, Homo sapiens
gugucugggcggacagcugc

SEQ ID NO: 139 - RNA, 21 nt, Homo sapiens
guguugaaacaaucucuacug

SEQ ID NO: 140 - RNA, 22 nt, Homo sapiens
agaaggaaauugaauucauuua

SEQ ID NO: 141 - RNA, 11 nt, Homo sapiens
ucuuaacgcaa

SEQ ID NO: 142 - RNA, 10 nt, Homo sapiens
accuguuagu

SEQ ID NO: 143 - RNA, 23 nt, Homo sapiens
ccuguuguuuuccuugacaaucu

SEQ ID NO: 144 - RNA, 75 nt, Homo sapiens
gcugcagagucccccccgguugccggcaagaccgcacaggcaaggccgaugacucugugcccacuccuguuguuu

SEQ ID NO: 145 - RNA, 75 nt, Homo sapiens
uccuugacaaucuacugugccacugugcuacuaacucuuguuuacaaaauuugauucuaaguugaauugcuucau

SEQ ID NO: 146 - RNA, 21 nt, Homo sapiens
uuucucuggccaagugacacu

SEQ ID NO: 147 - RNA, 21 nt, Homo sapiens
caaucuacugugccacugugc

SEQ ID NO: 148 - DNA, 3991 bp, Homo sapiens
agtcgcgcgggcagggctccgggcgcgagctgggctccttctgcaccacattcagcggctgccaagaggagccgacgggcgctcgcaggctcagcgcgcgctgcccgcggcaggacccggccgcctccgccgccgccgccgcccctaagcctcccgaagccatggccgggctcggccaccccgccgccttcggccgggccacccacgccgtggtgcgggcgctacccgagtcgctcggccagcacgcgctgagaagcgccaagggcgaggaggtggacgtcgcccgcgcggaacggcagcaccagctctacgtgggcgtgctgggcagcaagctggggctgcaggtggtggagctgccggccgacgagagccttccggactgcgtcttcgtggaggacgtggccgtggtgtgcgaggagacggccctcatcacccgacccggggcgccgagccggaggaaggaggttgacatgatgaaagaagcattagaaaaacttcagctcaatatagtagagatgaaagatgaaaatgcaactttagatggcggagatgttttattcacaggcagagaattttttgtgggcctttccaaaaggacaaatcaacgaggtgctgaaatcttggctgatacttttaaggactatgcagtctccacagtgccagtggcagatgggttgcatttgaagagtttctgcagcatggctgggcctaacctgatcgcaattgggtctagtgaatctgcacagaaggcccttaagatcatgcaacagatgagtgaccaccgctacgacaaactcactgtgcctgatgacatagcagcaaactgtatatatctaaatatccccaacaaagggcacgtcttgctgcaccgaaccccggaagagtatccagaaagtgcaaaggtttatgagaaactgaaggaccatatgctgatccccgtgagcatgtctgaactggaaaaggtggatgggctgctcacctgctgctcagttttaattaacaagaaagtagactcctgagctgcagagtcccccccggtagccggcaagaccgcacaggcaaggccgatgactctgtgcccactcctgttgttttccttgacaatctactgtgccactgtgctactaactcttgtttacaaaatttgattctaagttgaattgcttcattcaacacccccaccctccctcccctcgaggtggtacctaagctgtggatttgctaaatgaattaagcaacctagaagatacagagctaatgaattatcaaaatgtgattaatcccagtaaggaaacactcatttagtgtctgtatttttggtgtgaaaattatttagttgccagtatattctgaagaatgtcttcttgatcagtcagataagcttgctttttttttttttttttttcatgaatcatgtttggttcctgtgaaagtccctggtccagggatcctcctcctttctcttttacttctgaattctgaaattcagttagttacttttgcctttcgctcttctatcacagccaccttgaccttgggtaaaacccaaggtctttccttctggctaccttcctgcaggtccaccctgtctgccattggtctcctctgcctctgactacatctgccaccaacaaccctcccctcacccctgccaggggcagacaggcttctcagcagaactgtgactgaaatcagagctgctgtctggggcagtgttaactacacagaggcacatcctgacagggtttgccccagagatctaaattccagaaggagggcaccacacctaggaaggtaaatccagtatcagaaggttgctaaaagattaaagatcaagaagcttggaaacatcccatgggtacaatgtcttagaaagtctttaagtcacataccatgaatttttgcttcattactgaccatatatgaccttggaggaactcttttttttttttccttctactcatttctgtttccacctaccctgactcaccgtatttccagtcttctacccctgcagttatcctagtccagcaaagtcatttctttcaaaagagacatcatgtctgaaaataattactggtagtctaatatgagccagagtaaacagctcctcatggtcaatgaacatgttcaggaagcgatcaccttgatgcttgaacccaaccccagacagtggacaattctactttgaaatatccgtgaatatttactgtgggatccaatttaaacttctttcttctctagcctttaaattacacaactttgaactgacacggatctcttacaaagaacaatgcggcactgaaggaagagatgattcctttactcaaacctgcaggaatcagcctattaacaggcaggggaaacggtactttccaatgaatggtaactgatccaggcacattatcacacttcctagtcatctccacctttcctgtattgcctgtggcttgttgtttaagattaagaatcaaagagattaagaagtatcacttcaaatcttgctctgctcacttctatgtttgcagtcaaattattccttatgttggtgacctaaagagaattactttcattcatttcatttcccccgtagcagatggaaggagaaacctctgagaaaatgaaaacatccttaaccactatctttcccttttatttgattattttatgtcagaaatttgcaaaagtttttttctcctccttctcttccttgttgcttaactttttaattcatgccatatgcagatatccaattatgtgcatcctgtgaataaaccacgtcttggtcactgtcatattttgaaccatctcatcagagatgaataatatctttttaccagagagagaacgaatgttagccacatgcccaagttaacaaagaaaaaatgttctcaaggttgtccttttgggttaaatctggcccttccttggcaaaagcaaaaattctccctgtgagagctcaacatctcaaatacaaccacaggaaaaatggcccaatctgccagtttaggcttaccagcatataatttttaatatctttacttctatcatcccaaatcaaagaactcttctctattatgtttaatcaattgcaagcaaatagatttttctttgtaacaatttgttctgcagaaggctgtttttcacttttcctttcttttgcttctttctgtctttccttctcttttgtctggagaaatcacttagactctgtgtgcctcttctacattgcattctgctctgctatgttacctgctaggctggcttctttggactccctatatgattgatgatgtgaaaacctaaattacttgcagcatagtattacttctttgatgttctcattagcataatgttatttttgaaaaggaaagatactatcacataagttttcctcatctgttgtgatatacaccaatggataaactaacggaaactgctttttgacattaaaagacaggagaaattatatttaactaagtaaaagttaagtcagaattacttgggtgatgtgattcaatttagttaaaggatgatatagagaaaatacattatttagcattatttcttcagctataatgaattgctatagaaatcaggcagatctttctaatgtgtattgattggtcttttcagctactctgaacagattactaaggccatctcctcatctctaagggagaaaaatagtctgtagatgaataatgtaaggtaaagagttgcatgtcagtctttgtaattatttacactttaactttctccagaactcagacatgatttcaacatggtgttagatttgtgcattttattttcctgaccacctcattccagccaatgtatggttatccactctgtgtgccaaaaccaatcatgcctttcacggccctttagttcagagaagttctgcactgatttttagtctcttgatgtctcaatcttacatgtataccaatcacaatggaataaagtgttgagttgtactgtgaaaaaa

(19) United States
(12) Patent Application Publication
(10) Pub. No.: US 2016/0145615 A1
(43) Pub. Date: May 26, 2016

(54) AGONISTS OF DDAH1 FOR TREATING ENDOTHELIAL DYSFUNCTION

(71) Applicant: UCL BUSINESS PLC, London (GB)

(72) Inventors: Rajeshwar MOOKERJEE, London (GB); Gautam MEHTA, London (GB)

(21) Appl. No.: 14/899,247

(22) PCT Filed: Jun. 17, 2014
(86) PCT No.: PCT/GB2014/051867

§ 371 Date: Dec. 17, 2015

(30) Foreign Application Priority Data
Jun. 17, 2013 (GB) 1310755.2

(51) Int. Cl. C12N 15/113; C12Q 1/68; A61K 31/713

(52) U.S. Cl. CPC C12N 15/113; A61K 31/713; C12Q 1/6883; C12N 2310/141; C12N 2320/30; C12Q 2600/178; C12Q 2600/136

(57) ABSTRACT

The present invention derives from the finding that expression of DDAH1 is heavily post-transcriptionally regulated by microRNAs. By preventing or blocking the interaction between such microRNAs and the DDAH1 mRNA, the production of DDAH1 protein can be increased. This has utility in the prevention or treatment of diseases and disorders that are associated with reduced DDAH1 levels or increased ADMA levels, such as diseases or disorders that are characterised by endothelial dysfunction.

### FIELD OF THE INVENTION

The present invention relates to methods that target the expression of DDAH1. In particular, the invention relates to methods that target the interaction of particular miRNAs with the 3′ UTR of the DDAH1 gene and regulate its expression. These methods can be used to alter the expression of DDAH1 in vivo and to target disorders in which DDAH1 is involved, such as disorders characterised by endothelial dysfunction.

### BACKGROUND TO THE INVENTION

Dimethylarginine dimethylaminohydrolase (DDAH) is an enzyme found in all mammalian cells. Two isoforms exist, DDAH1 and DDAH2, with some differences in tissue distribution of the two isoforms. The enzymes degrade methylarginines, specifically asymmetric dimethylarginine (ADMA) and NG-monomethyl-L-arginine (MMA). DDAH2 has no significant ADMA metabolising effect. In contrast, DDAH1 drives over 90% of the metabolism of ADMA.

The methylarginines ADMA and MMA inhibit the production of nitric oxide synthase. Accordingly, DDAH is important in removing methylarginines, generated by protein degradation, from accumulating and inhibiting the generation of nitric oxide.

WO 2011/030103 teaches that decreased levels of DDAH1 are associated with increased portal pressure and relates to methods for reducing portal blood pressure by administering to a subject in need an agonist of DDAH1.

### SUMMARY OF THE INVENTION

The present invention derives from the finding that expression of DDAH1 is heavily post-transcriptionally regulated by microRNAs. In particular, the inventors have determined that miRNAs 128 and 219 can independently regulate DDAH1 expression, but may also act in combination with other microRNAs. By preventing or blocking the interaction between such microRNAs and the DDAH1 mRNA, the production of DDAH1 protein can be increased. This has utility in the prevention or treatment of diseases and disorders that are associated with reduced DDAH1 levels or increased ADMA levels, such as diseases or disorders that are characterised by endothelial dysfunction.

WO 2010/120969 relates to the possible effects of miR-30 on cardiac hypertrophy (cardiac myocytes) and heart failure (calcium regulation) whilst also suggesting the modulation of normal endothelial function, i.e. regeneration and maintenance of normal endothelial function, such as NO generation, to maintain coagulation and immune function. However, it does not concern conditions associated specifically with endothelial dysfunction.

Chen et al., (2013) PLOS One 8(5) e64148 reports that, under conditions of oxidative stress, 4-hydroxynonenal (4-HNE), a major active product formed following lipid peroxidation, increases ADMA levels in cultured HUVEC cells via miR-21 and downregulates both DDAH1 and DDAH2. The inventors have however determined that DDAH1 can be regulated by a variety of microRNAs, regardless of oxidative stress. Moreover, in conditions such as cirrhosis induced portal hypertension, there is increased DDAH2 despite low organ expression of DDAH1 which is at variance with that reported by Chen

The present invention therefore relates to methods for the treatment of diseases characterised by elevated levels of ADMA such as diseases characterised by endothelial dysfunction. In accordance with the present invention, this is achieved by administering an agent that targets the 3′ UTR of DDAH1, in particular an agent that targets miRNA that binds in that 3′ UTR, or targets the UTR binding site.

Accordingly, the present invention provides a method of treating or preventing a disease or disorder characterised by endothelial dysfunction comprising administering to a subject in need thereof an agonist of DDAH1, wherein said agonist prevents, inhibits or reduces the microRNA mediated repression of DDAH1 protein translation from DDAH1 mRNA.

Said agonist may lead to: (a) increased expression of DDAH1 protein in the subject; and/or (b) increased levels of DDAH1 in the subject.

Said microRNA may be miR-128 or miR-219. Said microRNA may be any of the microRNAs described herein, such as any of the mature miRs shown in Table 1, or any combination of these.

Said agonist may bind to said microRNA. For example, said agonist may be a nucleic acid molecule comprising a sequence that is complementary to at least a part of said microRNA. Said agonist may be a nucleic acid molecule that hybridises to said microRNA. Said agonist may bind to said microRNA and thereby prevent the microRNA from interacting with the DDAH1 mRNA.

The disease or disorder may be characterised by increased levels of asymmetric dimethylarginine (ADMA). For example, the subject may have coronary heart disease, peripheral vascular disease, chronic kidney disease, hypertension such as systemic hypertension, pulmonary hypertension, renovascular hypertension, portal hypertension or pregnancy induced hypertension/pre-eclampsia, raised inter-cranial pressure, stroke or chronic liver disease.

The invention also provides a method of identifying an agent suitable for use in treating portal hypertension, the method comprising determining whether a test agent is capable of binding to miR-128 or miR-219. Such a method may optionally further comprise a step of contacting a cell or tissue comprising DDAH1 mRNA and said microRNA with said test agent and determining whether the presence of the test agent leads to an increase in the amount of DDAH1 protein that is produced in the cell or tissue.

### BRIEF DESCRIPTION OF THE DRAWINGS

FIG. 1 reports on the 14C-citrulline counts per minute per milligram of protein. This is a measure of eNOS activity. Experiments were carried out in bile-duct ligated (BDL) or sham treated rats. The bile duct ligated rats were treated with either infliximab (BDL+infliximab) or a vehicle (BDL). This Figure shows that bile duct ligation markedly reduces eNOS activity, but that subsequent treatment with infliximab restores eNOS activity towards similar levels to those seen in the sham animals.

FIG. 2 reports ADMA levels in liver tissue in sham or BDL rats. The BDL rats were treated either with infliximab (BDL+INF) or with vehicle (BDL). Bile duct ligation led to a significantly elevated tissue ADMA concentration compared with sham animals. However ADMA levels were substantially reduced by treatment with infliximab. It is of interest that the relative concentration/accumulation of ADMA in the hepatic tissue in BDL animals is far greater than plasma ADMA concentration differences compared to sham animals.

FIG. 3 illustrates the markedly reduced expression of the DDAH1 isoform in livers of BDL rats. Upon treatment with Infliximab, the DDAH1 expression levels were restored towards sham levels.

FIG. 4 reports portal pressure in sham or BDL rats. Portal pressure was markedly increased in BDL cirrhotic rats compared to normal sham portal pressures (14±0.7 vs. 5.5±0.3 mmHg). Following intervention with Infliximab, this was reduced by more than 30% (9.5±0.6 mmHg).

FIGS. 5 and 6 report on hepatic eNOS activity (FIG. 5) and hepatic eNOS protein expression (FIG. 6). It was found that eNOS activity was significantly decreased in BDL animals compared to sham (*-p<0.05) despite increased eNOS protein expression (**-p<0.01). Following treatment with INT-747, eNOS activity reverted to sham levels (*-p<0.05), with similar normalisation of eNOS protein expression (*-p<0.05).

FIGS. 7 and 8 report on hepatic ADMA protein expression (FIG. 7) and hepatic DDAH1 protein expression (FIG. 8). It was found that ADMA expression was significantly increased in BDL animals (**-p<0.01), concomitant with significantly reduced DDAH1 protein expression (**-p<0.01). Following INT-747 administration DDAH1 expression increased significantly (**-p<0.01) with a significant reduction in ADMA (*-p<0.05) compared with BDL alone.

FIG. 9 reports portal pressure in sham or BDL rats. Portal pressure was significantly increased in BDL rats compared to sham (***-p<0.0001). Following INT-747 treatment there was a 30% reduction in portal pressure when compared to BDL+vehicle (**-p<0.01).

FIG. 10 reports the effects of hydrodynamic gene delivery of DDAH1 expressing plasmid into BDL rats leading to increased DDAH1 mRNA (A) and decreased portal pressure (B) relative to control plasmid.

FIG. 11 shows the expression of DDAH1 in sham treated and bile duct ligated (BDL) rats. The left hand panel shows the expression of DDAH1 protein as assessed by Western blot. The right hand panel shows the expression of DDAH1 mRNA as measured by quantitative PCR.

FIG. 12 shows the results of bioinformatic analysis of the DDAH1 3′ untranslated region. FIG. 12A is a screenshot showing predicted DDAH1 3′UTR binding sites obtained using TargetScan (v5.2). FIG. 12B is a screenshot showing predicted DDAH1 3′UTR binding sites using miRanda (microrna.org August 2010 release). FIG. 12C is screenshot showing predicted DDAH1 3′UTR binding sites obtained using TargetScan (v6.2). FIG. 12D is a screenshot showing predicted DDAH1 3′UTR binding sites using miRanda (microrna.org August 2010 release).

FIG. 13 shows the hepatic levels of different miRNAs in sham treated or BDL rats. FIG. 13A shows the expression of miR-128 and FIG. 13B shows the expression of miR-30a.

FIG. 14 is a pictorial representation of one group of predicted miRNA binding sites on the 3′ UTR of DDAH1. The boxed area towards the middle is a 52 bp region containing predicted binding sites for miRs 219, 128 and 30.

FIG. 15 shows that the 52 bp sequence shown in FIG. 14 is highly regulatory to the same degree as the entire DDAH1 3′UTR and that mutation of the miR-128 binding site causes the loss of repression of luciferase expression. This repression is greater than with mutation of miR-219 or miR-30a seed sequences.

FIG. 16 shows a Western blot for DDAH1 protein expression in HEK293T cells 72 hours after transfection with synthetic miRNAs, Synthetic miRNA mimics for miR-128 (left panel), miR-30a (centre panel) and miR-219 (right panel) all significantly reduced DDAH1 protein expression.

FIG. 17 shows the mRNA sequence for DDAH1. The coding sequence is shown in bold text, starting at nucleotide 162 and ending at nucleotide 1019. The 3′UTR sequence is underlined, starting at nucleotide number 1020.

FIG. 18 shows the relative expression of DDAH1 mRNA in Sham and BDL (Cirrhosis) rat Liver. BDL rats had significantly higher portal pressure than sham rats, confirming the development of cirrhosis and portal hypertension. The level of DDAH1 mRNA was increased (non-significantly) between BDL and sham rats despite previous data demonstrating lower DDAH1 protein expression, suggesting post transcriptional regulation. The level of miR-128 is markedly elevated in BDL rats compared to sham animals. The 2-plex ISH data suggests a co-localization of miR-128 and DDAH1 distributed widely throughout the cirrhotic liver specimens as shown in the merged figure.

### DETAILED DESCRIPTION OF THE INVENTION

The inventors have found that expression of the DDAH1 isoform of dimethylarginine dimethylaminohydrolase (also referred to herein as DDAH I) is significantly reduced in established models of cirrhosis (such as bile duct ligated rats—BDL, compared to sham rats). Expression of the DDAH1 isoform is found to be further decreased in the context of superadded inflammation/infection (through endotoxin challenge). Moreover, the inventors have found that agonism of DDAH1, either by increasing its activity or by increasing its expression, leads to an increase in eNOS activity, a decrease in levels of methylarginines (such as ADMA) and a significant lowering in portal pressure. This confirms that agents that increase the expression or activity of DDAH1 can be used therapeutically to decrease levels of ADMA, and to treat or prevent disorders that are characterised by increased ADMA levels such as disorders that are characterised by endothelial dysfunction.

The inventors have further investigated the post-transcriptional regulation of DDAH1 expression. They have found that the expression of DDAH1 in vivo is heavily regulated at the mRNA level. In particular, they have found that DDAH1 mRNA is heavily post-transcriptionally regulated by endogenous microRNAs (miRNAs, miRs). This means that traditional approaches to increase DDAH1 expression, such as pharmacological and gene therapy methods to promote expression from the DDAH1 gene, may have limited effects as the expression of DDAH1 protein will still be regulated post-transcriptionally by microRNAs. It would therefore be advantageous to be able to target this miR regulation of the DDAH1 mRNA. By reducing the effects of miRs on the DDAH1 mRNA, expression of the DDAH1 protein can be increased. This approach may be used alone, to increase expression of the DDAH1 protein from endogenously transcribed DDAH1 mRNA, or it may be used in combination with other approaches intended to increase the expression or activity of DDAH1.

The present invention therefore lies in the augmentation of DDAH1 protein expression by targeting the interaction between miR and DDAH1 mRNA. This approach can be used to increase DDAH1 expression and thereby to decrease levels of ADMA in vivo. This has utility in the prevention or treatment of disorders that are characterised by increased ADMA levels, such as disorders characterised by endothelial dysfunction.

### DDAH1 Agonists

The present invention relates to the agonism of DDAH1 protein expression. An agonist of DDAH1 for use in the present invention may be a compound or molecule that increases the amount of DDAH1 protein that is expressed in a cell. The agonist of DDAH1 may be a compound or molecule that reduces the inhibition of DDAH1 protein translation from DDAH1 mRNA by miRs.

MicroRNAs (miRs) are a class of post-transcriptional regulators. They are short ˜22 nucleotide RNA sequences that bind to complementary or near-complementary sequences in the 3′ UTR of target mRNAs, usually resulting in the silencing of the mRNA. Silencing may be achieved by, for example, translational repression or target degradation and gene silencing.

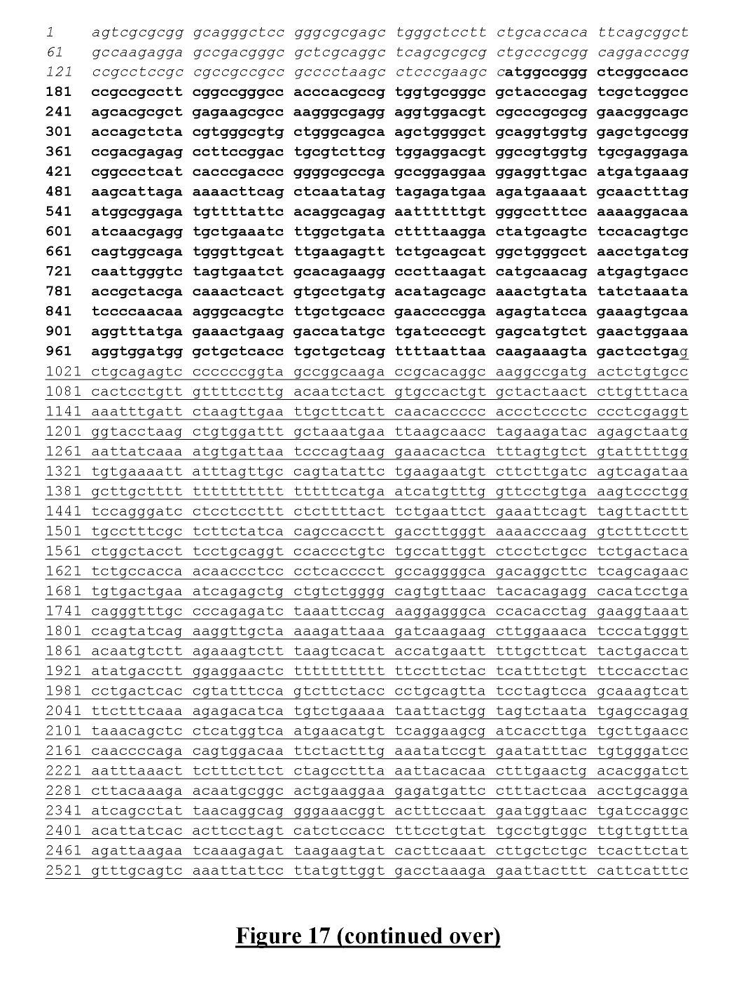

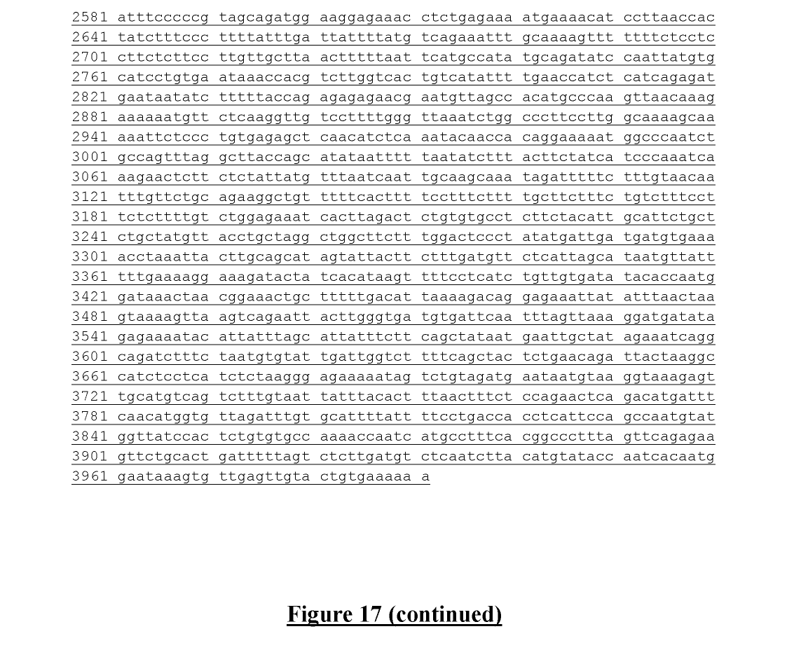

The sequence of DDAH1 mRNA can be found in GenBank entry NM_012137, such as is version number NM_012137.3 dated 9 Dec. 2012. References to particular nucleotides or regions in the 3′UTR if DDAH1 herein are made with reference to that version of NM_012137. The mRNA sequence for DDAH1 is shown in FIG. 17. The 3′UTR sequence is underlined in FIG. 17.

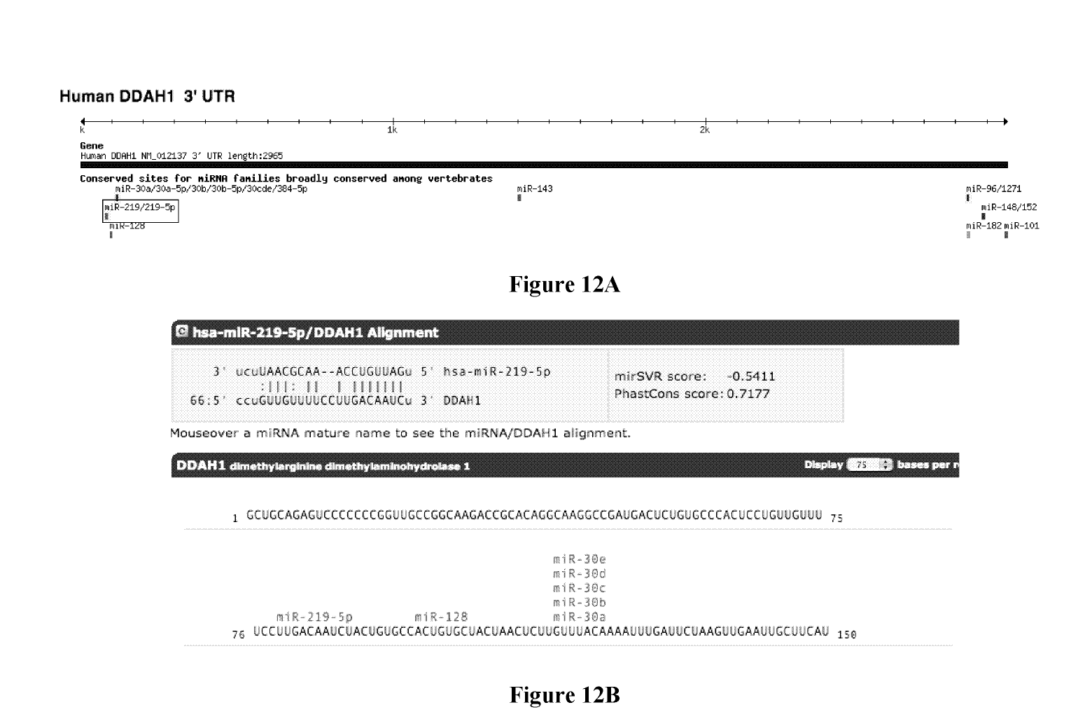

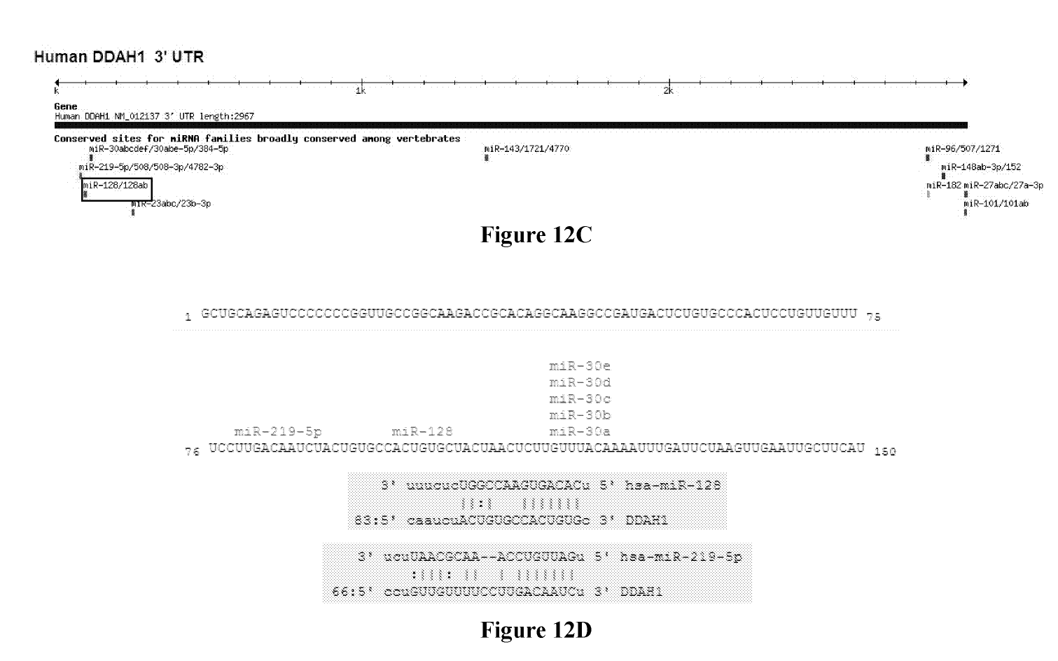

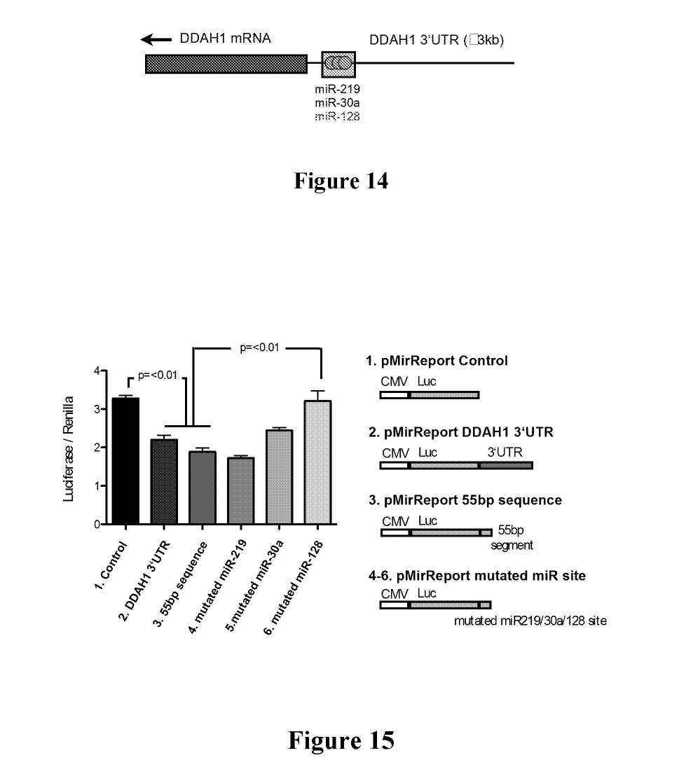

The agonist may act by blocking the interaction between one or more miRs and DDAH1 mRNA. The agonist may act by blocking the interaction between one or more miRs and the 3′ UTR of DDAH1 mRNA. The agonist may act by blocking the interaction between one or more miRs and the 52 bp sequence of the 3′UTR of DDAH1 mRNA that is described in FIG. 12 and FIG. 14.

The 52 bp sequence may be found at nucleotides 1091 to 1142 of the DDAH1 mRNA sequence (FIG. 17). The 52 bp fragment has the following sequence:

```
g t t t t c c t t g a c a a t c t a c t g t g c
                                          miR-219
c a c t g t g c t a c t a a c t c t t g t t t a
    miR-128 miR-30
c a a a
```

As shown above, this 52 bp sequence includes binding sites for miR-219, miR-128 and miR-30.

The agonist may act by blocking the interaction between miR-219 and/or miR-128 and the 3′UTR of DDAH1. The agonist may act by blocking the interaction between miR-219 and/or miR-128 and the 52 bp sequence of the 3′UTR of DDAH1 mRNA that is described in Examples 7 and 9.

Blocking the interaction between an miR and the 3′UTR or a part thereof as indicated above encompasses any effect that results in the miR not having an inhibitory effect on the expression of the DDAH1 protein. This may be, for example, by preventing the production of the miR, by reducing the amount of miR that is present, by preventing binding of the miR to the 3′UTR, by sequestering or otherwise binding the miR and thereby preventing it from binding to the 3′UTR or by binding or otherwise blocking the binding site for the miR in the 3′UTR thereby preventing the miR from binding to the 3′UTR.

The overall effect of the agonist is therefore to prevent the suppression of DDAH1 protein production that is caused by miR binding in the 3′UTR of the DDAH1 mRNA molecule.

The agonist may act to prevent the effects of one or more miRs that normally bind in the 3′UTR of DDAH1 mRNA. For example, as illustrated in FIGS. 12 and 14, binding sites are believed to exist in the DDAH1 3′UTR for at least miR-219, miR-128, miR-30, miR-143, miR-96, miR-148, miR-182, miR-101, miR-21 and miR-210. The miR may be any of these miRs.

The miR in accordance with the present invention may be selected from the following list: miR-128, miR-219, miR-21, miR-210, miR-30, miR-508, miR-23, miR-143, miR-1721, miR-4770, miR-96, miR-507, miR-1271, miR-148, miR-152, miR-182, miR-27, miR-101, miR-765, miR-589, miR-1299, miR-595, miR-301, miR-548, miR-1261, miR-943, miR-635, miR-509, miR-548, miR-1231, miR-653 and miR-1252.

The agonist may act to prevent the effects of any one or more of the miRs listed herein on the expression of DDAH1 protein.

Preferably the miR is one that binds to the DDAH1 3′UTR in the 52 bp region identified in Examples 7 and 9 and as shown above. Preferably the miR is miR-219 or miR-128, or both of these miRs. The miR-219 may be miR-219-5p.

Sequences for different miRs may be found at miRbase.org. References herein to miRs include the mature sequence. References to miRs include all transcripts of that miR. For example references herein to miR-30 encompass any one or more of miR-30a, miR-30b, miR-30c, miR-30d and miR-30e. References herein to miRs include both possible strands of those miRs, for example references herein to miR-219 include miR-219-5p and miR-219-3p. A number of mature miR sequences that may be used in accordance with the invention are set out in Table 1 below. The miRs in accordance with the invention are preferably human miRs.

Any one, two or three of these miRs may be combined with any other miRs that would normally bind to the DDAH1 3′ UTR.

Preferably miR-128 and/or miR-219 can be combined with any combination of the other miRs that would normally bind to the DDAH1 3′ UTR.

Preferably miR-128 and/or miR-219 can be combined with any combination of the other miRs that are set out in Table 1 below.

In particular, miR-128 and/or miR-219 can be combined with one or more of miR-30, miR-508, miR-23, miR-143, miR-1721, miR-4770, miR-96, miR-507, miR-1271, miR-148, miR-152, miR-182, miR-27, miR-101, miR-765, miR-589, miR-1299, miR-595, miR-301, miR-548, miR-1261, miR-943, miR-635, miR-509, miR-548, miR-1231, miR-653 and miR-1252; or with 2, 3, up to 5, up to 10 or more, or all of these.

The miR or combination of miRs may be specific to the DDAH1 3′UTR. That is, the agonist may be selected to act on an miR or combination of miRs that are only involved in the regulation of DDAH1 expression, not the expression of other genes, or that have a greater effect on the expression of DDAH1 than on the expression of other genes.

The agonist may have an effect in any tissue in which DDAH1 mRNA is expressed. The agonist may be capable of acting in any tissue in which DDAH1 is expressed.

The agonist may have a preferential effect or may only be capable of acting in one or more specific tissue types. The agonist may be targeted to one or more specific tissue types. The tissues where the agonist is capable of acting are preferably those where an increase in DDAH1 expression is desired, such as those in which a decrease in ADMA levels is desired. For example, the tissue may be a tissue that exhibits, or is susceptible to, endothelial dysfunction. The agonist may have its function in one or more of the liver, kidney, heart or vascular system of the patient. Preferably the agonist leads to an increase in DDAH1 in one or more of the liver, kidney, heart or vascular system of an individual to whom the agonist is administered. The agonist may act preferentially in one or more of the liver, kidney, heart or vascular system or may act at a number of locations including one or more of the liver, kidney, heart or vascular system. The agonist may be targeted to one or more of the liver, kidney, heart or vascular system during administration as discussed further below.

Preferred agonists are those that increase the amount of DDAH1 protein in a cell or tissue by at least 10%, at least 20%, at least 30%, at least 40%, at least 50%, at least 60%, at least 70%, at least 80% or at least 90% compared to the amount seen in the absence of the agonist. For example, increases of these sizes may be seen in the one or more tissues or organs of a subject to whom the agonist has been administered.

The agonist may act specifically to agonise DDAH1. That is, the effect of the agonist on DDAH1 may be greater than any other biological effect of the agonist. Such an agonist may be specific to the expression of DDAH1, that is it may increase or maintain the expression of DDAH1 but not other proteins.

An agonist for use in accordance with the present invention may act on DDAH1 in preference to DDAH2 (also known as DDAH II). For example, an agonist of DDAH1 for use in accordance with the present invention may have one or more of the characteristics of a DDAH1 agonist as described herein, but may not have such characteristics in relation to DDAH2, or may have such characteristics to a lower level in relation to DDAH2 when compared to DDAH1. For example, an agonist that increases the expression or amount of DDAH1 may not increase the expression or amount of DDAH2, or may increase the expression of DDAH2 to a lesser extent, such as a lower percentage increase, than its effect on DDAH1. The agonist may act on miRs that do not bind in the 3′UTR of DDAH2. The effect of an agonist may be assessed by looking for the presence of DDAH1 protein or by looking for an increase in activity of DDAH1. The primary function of DDAH1 is the enzymatic degradation of methylarginines such as asymmetric dimethylarginine (ADMA). An agonist of the present invention may therefore act to increase the degradation of methylarginines by DDAH1, such as the degradation of ADMA. An agonist of the invention may act to increase the amount of DDAH1 and thereby lead to a decrease in the level of methylarginines such as ADMA, preferably in the liver. Agonist activity may therefore be identified by the ability to cause a decrease in ADMA levels, for example in the liver.

Methylarginines such as ADMA inhibit the production of nitric oxide synthase (NOS). Accordingly, an agonist of the present invention may act to increase the activity, function or amount of DDAH1 and thereby increase levels of NOS, preferably in the liver. Agonist activity may therefore be identified by the ability to increase levels of NOS, for example in the liver.

Agonists for use in accordance with the present invention may act directly or indirectly on the one or more miRs as described herein.

Direct agonists are agents whose activity is directly on the one or more miRs. For example, direct agonists may be agents that act directly on the miR molecule(s) to decrease or to prevent their activity. A direct agonist may be an agent that causes or increases the degradation of the miR(s) or decreases their half-life in vivo. A direct agonist may be an agent that reduces the stability of the miR(s). A direct agonist may decrease the amount of the miR(s) that is present, for example by preventing the production of the miR(s), by preventing the miR(s) from reaching the location of the DDAH1 3′UTR, by degrading miR(s) that are present or by reducing the stability of the miR(s). A direct agent may bind to the miR(s) and thereby prevent them from interacting with the 3′UTR. A direct agonist may be an agent that acts on the DNA sequence that encodes the miR(s) to prevent or reduce the transcription of the miR(s).

Indirect agonists are agents whose activity leads to agonism of DDAH1 by preventing the miR(s) from suppressing expression of DDAH1 protein, but which do not act directly on the miR(s) of interest. For example, indirect agonists include agents that have an effect on the region of the DDAH1 3′UTR that is bound by the miR(s) and prevents the binding of the miR(s) in that region. For example, the agonist may bind to an adjacent or overlapping part of the 3′UTR and thereby prevent the miR(s) from accessing their normal binding sites.

An agonist for use in the present invention may bind to the miR(s) of interest and prevent them from binding to the 3′UTR of DDAH1. An miR generally binds to a complementary or near-complementary nucleic acid sequence in the 3′UTR by hybridisation. The agonist may therefore comprise a nucleic acid molecule such as an oligonucleotide or polynucleotide that is capable of binding by hybridisation to the miR. The agonist may comprise a nucleic acid that is complementary to all or part of the miR sequence of interest. The agonist may therefore be an antisense oligonucleotide. A suitable antisense oligonucleotide may be, for example, a locked nucleic acid (LNA), a 2-O-methyl oligonucleotide, a 2-O-methoxyethyl oligonucleotide or an antagomir as described in more detail below. A suitable agonist may be or may comprise a DNA or RNA molecule or a DNA-RNA hybrid, as long as it is capable of binding to the miR of interest. The nucleic acid may be a single stranded or double stranded nucleic acid, but is preferably a single stranded sequence that is complementary to all or part of the miR of interest.

The agonists of the invention may therefore comprise nucleic acid sequences that are complementary to a region of at least one miR of interest. The nucleic acid sequence may be complementary to 10, 11, 12, 13, 14 or 15 consecutive nucleotides of the miR sequence.

The nucleic acid may be an oligonucleotide. The length of the oligonucleotide is not fixed, as long as the oligonucleotide is capable of binding to at least one miR by complementary binding. The oligonucleotide may be at least 20, 21, 22, 23, 24, 25, 26 or 27 nucleotides in length, preferably at least 23, 24, 25 or 26 nucleotides in length, especially 25 nucleotides in length. The oligonucleotide may comprise a nucleic acid sequence that is complementary to a sequence of the miR(s) and may further comprise additional nucleotides at the 5′ and/or 3′ ends of that complementary sequence. The oligonucleotide may consist of a sequence that is complementary to a sequence of the miR(s).

The nucleic acid may be a polynucleotide, such as a polynucleotide comprising an oligonucleotide sequence as described herein. The polynucleotide may be at least 30, at least 35, at least 40, at least 50, at least 55, at least 60 or more nucleic acid residues in length. For example, the polynucleotide may be up to 30, up to 35, up to 40, up to 50, up to 55, up to 60, up to 70, up to 80, up to 90 or up to 100 nucleic acid residues in length.

The nucleic acid may bind selectively to the miR of interest, i.e. it binds to the miR of interest but not to other miRs that may be present in the same cell. The nucleic acid may therefore comprise a section of nucleic acid sequence that is complementary with enough of the miR sequence to ensure that binding occurs only to the miR of interest, or preferentially to the miR instead of other miRs that may be present.

The nucleic acid may bind to multiple miRs of interest. For example, binding sites for miRs 219, 128 and 30 have been found within a 52 bp region of the DDAH1 3′UTR.

The nucleic acid may therefore comprise any one, two or three of these binding sites. For example, the nucleic acid may have the sequence of the 52 bp region of the DDAH1 3′UTR described herein or a fragment thereof comprising one, two or three of the binding sites (for miR-219 and/or miR-128 and/or miR-30) as illustrated above.

The nucleic acid may therefore comprise sequences that are complementary to all or part of more than one miR. The sequences that bind to different miRs may be separate within the nucleic acid, or may be overlapping.

The agonist for use in the present invention may comprise all or part of the 3′UTR sequence of DDAH1. Such an agonist may be an oligonucleotide that is or that comprises a fragment of the 3′ UTR sequence of DDAH1, such as a fragment that comprises a binding site for the miR(s) of interest. Such an agonist may be a longer polynucleotide comprising such a binding site, such as a longer fragment of the DDAH1 3′UTR or a longer polynucleotide comprising such an oligonucleotide. A suitable polynucleotide may consist of or comprise the 52 bp region of the DDAH1 3′UTR described in Example 5, 7 and 9. A suitable oligonucleotide or polynucleotide may comprise or consist of a fragment of that 55 bp region, as long as the fragment is capable of binding the miR(s) of interest.

For example, as illustrated above, within the 52 bp region of the DDAH1 3′UTR described in Examples 7 and 9, binding sites for miR-219, miR-128 and miR-30 have been identified.

The binding site for miR-219 is at the sequence gacaatc. A nucleic acid may therefore be used that comprises the sequence gacaatc in order to bind to miR-219. The nucleic acid may be a fragment of the 52 bp sequence that comprises the sequence gacaatc.

The binding site for miR-128 may be found at the nucleotide sequence cactgt. A nucleic acid may therefore comprise the sequence cactgt in order to bind to miR-128. The nucleic acid may be a fragment of the 52 bp sequence that comprises the sequence cactgt. The nucleic acid sequence tgtttac may be bound by miR-30. A nucleic acid comprising the sequence tgtttac may therefore be used to bind to miR-30. The nucleic acid may be a fragment of the 52 bp sequence that comprises the sequence tgtttac.

Similar nucleic acid sequences may be determined for other miRs as described herein. A number of mature miR sequences are set out in Table 1 below. Further information about the miRs may be obtained from www.mirbase.org and the nucleic acid sequences bound by these miRs may be determined using routine techniques.

Nucleic acids containing nucleotide sequences which are perfectly complementary to a portion of the target miR(s) may be employed. In some instances, sequence variations that might be expected due to genetic mutation, strain polymorphism, or evolutionary divergence may be present. For example, oligonucleotide reagent sequences with insertions, deletions, and single point mutations relative to the target sequence may also be effective. Greater than 70% sequence identity (or complementarity), e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity, between the oligonucleotide reagent and a target miR is preferred. Such sequence identity may be over the region hybridising to the miR or, for instance, over the whole oligonucleotide, though in some instances that may be the same thing.

Sequence identity, including determination of sequence complementarity for nucleic acid sequences, may be determined by sequence comparison and alignment algorithms known in the field. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions*100), optionally penalizing the score for the number of gaps introduced and/or length of gaps introduced.

The comparison of sequences and determination of percent identity between two sequences can be accomplished using a mathematical algorithm. The alignment may be generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

Alternatively, the alignment may be optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402.

The alignment may be optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

A suitable agonist may therefore be or comprise a nucleic acid such as an oligonucleotide or polynucleotide. The nucleic acid may, for example, be DNA or RNA or a DNA/RNA hybrid and is preferably RNA. The nucleic acid may comprise naturally occurring or modified nucleotides. The nucleic acid may comprise naturally occurring bonds between nucleic acid monomers or may comprise non-naturally occurring bonds or groups linking together adjacent nucleic acids.

The agonist may therefore be a synthetically produced nucleic acid molecule. A variety of such molecule types are known and some examples as described below. Any nucleic acid analog that is capable of binding specifically to a nucleic acid sequence, e.g. via a complementary nucleic acid sequence, may be used in the present invention. Some synthetic analogs confer advantages over naturally-occurring nucleic acid molecules, such as improved stability or improved strength of binding to a complementary sequence.

The nucleic acid may therefore comprise modifications. The nucleic acid may be modified by the substitution of at least one nucleotide with at least one modified nucleotide, ideally so that the in vivo stability of the nucleic acid is enhanced as compared to a corresponding unmodified nucleic acid. The modified nucleotide may, for example, be a sugar-modified nucleotide or a nucleobase-modified nucleotide. One, some or all of the nucleotides in the nucleic acid may be modified in one or more of the ways described herein. Different modifications may be present in different nucleotides.

The modified nucleotide may be a 2′-deoxy ribonucleotides such as 2′-deoxy guanosine or 2′-deoxy adenosine. The modified nucleotide may be a 2′-O-methylguanosine, 2′-O-methyl (e.g., 2′-O-methylcytidine, 2′-O-methylpseudouridine, 2′-O-methyluridine, 2′-O-methyladenosine, 2′-O-methyl) ribonucleotide. The modified nucleotide may be 2′-amino, 2′-thio or 2′-fluoro modified ribonucleotide. The modified nucleotide may be 2′-fluoro-cytidine, 2′-fluoro-uridine, 2′-fluoro-guanosine, 2′-fluoro-adenosine, 2′-amino-cytidine, 2′-amino-uridine, 2′-amino-adenosine, 2′-amino-guanosine, 2′-amino-butyryl-pyrene-uridine and 2′-amino-adenosine. In an additional instances, the modified nucleotide is selected from 5-iodo-uridine, ribo-thymidine, 5-bromo-uridine, 2-aminopurine, 5-methyl-cytidine, 5-fluoro-cytidine, and 5-fluoro-uridine, 2,6-diaminopurine, 4-thio-uridine or 5-amino-allyl-uridine.

The modification of the nucleotide may include: derivitization of the 5 position, for example 5-(2-amino)propyl uridine, 5-bromo uridine, 5-propyne uridine, 5-propenyl uridine; derivitization of the 6 position, for example 6-(2-amino)propyl uridine; derivitization of the 8-position for adenosine and/or guanosines, for example 8-bromo guanosine, 8-chloro guanosine, or 8-fluoroguanosine, Nucleotide analogs which may be employed include deaza nucleotides, e.g., 7-deaza-adenosine; 0- and N-modified (for instance alkylated, such as N6-methyl adenosine) nucleotides; and other heterocyclically modified nucleotide analogs. Examples of modifications to the sugar portion of the nucleotides which may be employed include the 2′ OH-group being replaced by a group selected from H, OR, R, F, Cl, Br, I, SH, SR, NH2, NHR, NR2, COOR, or OR, wherein R is substituted or unsubstituted C1-C6 alkyl, alkenyl, alkynyl, aryl etc.

The phosphate group of the nucleotide may be modified, such as by substituting one or more of the oxygens of the phosphate group with sulphur (for instance by employing phosphorothioates). Modifications may decrease the rate of hydrolysis of polynucleotides comprising the modified bases, for example by inhibiting degradation by exonucleases. In one preferred instance, the nucleic acid is resistant to ribonucleases. The nucleic acid may include modifications that promote such resistance, for example modification with a 2′-O-methyl group (e.g., 2′-O-methylcytidine, 2′-O-methylpseudouridine, 2′-O-methylguanosine, 2′-O-methyluridine, 2′-O-methyladenosine, 2′-O-methyl) and/or presence of a phosphorothioate backbone.

The nucleic acid may comprise phosphorothioate and 2′-O-methyl (e.g., 2′-O-methylcytidine, 2′-O-methylpseudouridine, 2′-O-methylguanosine, 2′-O-methyluridine, 2′-O-methyladenosine, 2′-O-methyl) modification. The modified nucleotide employed may be 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluraci 1,5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, or 2,6-diaminopurine.

The modified nucleic acid may include modifications to the phosphate backbone such as methyl phosphonates, methyl phosphonothioates, phosphoromorpholidates, phosphoropiperazidates and phosphoramidates. In one example, every other one of the internucleotide bridging phosphate residues may be modified as described. In another example, at least one, or all, of the nucleotides contain a 2′ lower alkyl moiety (e.g., C1-C4, linear or branched, saturated or unsaturated alkyl, such as methyl, ethyl, ethenyl, propyl, 1-propenyl, 2-propenyl, and isopropyl).

Other forms of nucleotide modifications may be employed, for example, locked nucleic acids. A locked nucleic acid (LNA) is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. One or more LNA nucleotides can be included in DNA, RNA or DNA-RNA hybrid molecules. Such molecules are synthesized chemically and are commercially available. The locked ribose conformation enhances base stacking and backbone pre-organization. This significantly increases the hybridization properties of oligonucleotides. A nucleic acid comprising at least one LNA may therefore be used as an agonist in accordance with the present invention in order to bind to one or more miR of interest and prevent them from binding to the 3′UTR in DDAH1 mRNA.

The agonist may be a morpholino. Morpholinos are synthetic polymeric molecules that are capable of binding to complementary sequences of RNA by standard nucleic acid base-pairing. A morpholino polymer lacks a pentose sugar backbone moiety, and more specifically a ribose backbone linked by phosphodiester bonds which is typical of nucleotides and nucleosides, but instead contains a ring nitrogen with coupling through the ring nitrogen. In such instances, the riboside moiety of each subunit of an oligonucleotide of the invention may be converted to a morpholine moiety (C₄H₉NO). Thus, structurally, morpholinos have standard nucleic acid bases, but those bases are bound to morpholine rings instead of deoxyribose or ribose rings and the bases are linked through phosphorodiamidate groups instead of phosphates.

Replacement of anionic phosphates present in naturally occurring polynucleotides with the uncharged phosphorodiamidate groups eliminates ionization in the usual physiological pH range, so morpholinos in organisms or cells are uncharged molecules. The entire backbone of a morpholino is generally made from these modified subunits. Morpholinos generally act by steric blocking, i.e. binding to a target sequence within an RNA and simply getting in the way of molecules that might otherwise interact with the RNA. A morpholino, a nucleic acid comprising a morpholino may therefore be used as an agonist in accordance with the present invention in order to bind to one or more miR of interest and prevent them from binding to the 3′UTR in DDAH1 mRNA. The modifications present in a morpholino, such as replacement of deoxyribose rings with morpholine rings and/or the replacement of phosphates with phosphorodiamidate groups, may be present in one or more nucleotides of a nucleic acid that is used as an agonist in accordance with the present invention.

The agonist may be an antagomir. Antagomirs are chemically engineered oligonucleotides that may be used to silence microRNA. An antagomir is a synthetic RNA that is complementary to the microRNA target with either mispairing at the cleavage site of Ago2 or a base modification to inhibit Ago2 cleavage. An antagomir may have a further modification, such as a 2′ methoxy group or a phosphothioate, to make it more resistant to degradation. Antagomirs are believed to inhibit miR by irreversibly binding the miR molecule. Antagomirs can therefore be used to constitutively inhibit the activity of specific miRNAs. An antagomir may therefore be used as an agonist in accordance with the present invention in order to bind to one or more miR of interest and prevent them from binding to the 3′UTR in DDAH1 mRNA.

A variety of other strategies for interfering with miRs may be used and are known in the art. For example, a viral strategy may be used to knockdown the relevant miR(s). For example, vectors such as AAV, lentivirus or adenovirus may be used to block miRs. A variety of approaches to blocking the interaction of miRNAs with their target sequences are discussed in van Rooij and Olson Nature Reviews Drug Discovery (2012) 11: 860-872. For example, pseudogenes are non-coding transcript that contain conserved miRNA binding sites that can act as decoys to interfere with miRNA activity. Anti-miRs are antisense oligonucleotides designed to target specific miRNAs. Anti-miRs can use various high affinity 2′ sugar modifications such as conformationally restricted nucleotides with 2′-O-methyl (2′-O-Me), 2′-O-methoxyethyl (2′-MOE), 2′-fluoro (2′-F) or locked nucleic acid (LNA) modifications, as discussed above. To increase nuclease resistance, these molecules may include phosphorothioate backbone linkages, whereby a sulphur atom replaces one of the non-bridging oxygen atoms in the phosphate group.

Any of the agonists described herein may be used to agonise DDAH1, i.e. to increase the amount of DDAH1 that is present. Preferably these agonising effects take place in the liver.

The agonists of the invention may act at, or be effective at, at a concentration (e.g., have an IC50) in the nanomolar range, for example, less than 1000 nm, for instance less than 500 nM, preferably less than 400 nM, more preferably less than 300, 250, 200, 150, 100, 75, 50, 25, 10, 5, 2 or 1 nM.

Some agents that are capable of agonising DDAH1 may be unsuitable for in vivo administration as part of a treatment as described herein. Some agents that are capable of agonising DDAH1 may have other unwanted effects on the patient. A physician will be able to balance for an individual patient whether those unwanted side-effects outweigh the potential benefits of the DDAH1 agonism described herein in order to select a suitable DDAH1 agonist for use as described herein.

An agonist of the invention may be administered in combination with one or more other therapies. For example, the agonist may be administered in combination with one or more other therapies that are intended to prevent or treat the same, or a related, condition. Where multiple therapies are used, they may be used simultaneously (e.g. administration in a single composition), sequentially or separately as part of an overall combined therapy.

Such a combined administration may comprise administration of a further agent that is intended to increase or maintain the amount or activity of DDAH1 or a further agent that is intended to decrease or prevent the amount or activity of ADMA.

For example, a method of the present invention may be combined with administration of an agent that increases the production of endogenous DDAH1. For example, the agent may act within the cells of the subject to enhance or stimulate the expression of DDAH1. Such an agent may be a transcription factor or enhancer that acts on the DDAH1 gene to promote gene expression. For example, such an agent may be a nuclear receptor such as the Farnesoid receptor agonist INT-747.

A method of the invention may be combined with administration of an agent that provides the cells of an individual with the ability to produce additional DDAH1. For example, the agent may be a vector that is capable of expressing DDAH1 such as an expression vector comprising a DDAH1 gene and other sequences as necessary for expression of that gene. Thus, DDAH1 may be provided by delivering such a vector to a cell and allowing transcription from the vector to occur. The agent may be a polynucleotide that is capable of expressing DDAH1 such as a vector comprising a DDAH1 gene and other sequences as necessary for integration of the DDAH1 gene into the host genome and to allow expression from that inserted DDAH1 gene sequence. Methods for gene delivery are known in the art. See, e.g., U.S. Pat. Nos. 5,399,346, 5,580,859 and 5,589,466. The polynucleotide may be expressed under the control of a suitable promoter. For example, expression of the DDAH1 polynucleotide may be targeted to the liver by using a liver specific promoter. Thus, the agent may be a polynucleotide or vector comprising a DDAH1 gene and a liver specific promoter.

### Screening Methods

The present invention also provides methods for the identification of agents suitable for use in the treatment or prevention of disorders characterised by endothelial dysfunction. For example, the invention provides methods for the identification of agonists of DDAH1 which reduce or prevent the inhibition of protein expression from the DDAH1 mRNA by one or more microRNAs. Agonists identified by this method may be agonists of DDAH1 having any of the characteristics or effects described above.

Accordingly, the invention provides a method of identifying an agent for use in the treatment of disorders characterised by endothelial dysfunction, the method comprising determining whether a test agent is capable of reducing or preventing the inhibition of DDAH1 protein expression by one or more miRs. For example, the method may involve determining whether a test agent is capable of binding to one or more miRs of interest or whether a test agent is capable of preventing or reducing the binding of one or more miRs to the 3′ UTR of DDAH1 mRNA. In such methods, the ability to bind to the relevant miR(s) and/or to prevent the effects of those miRs indicates that the test agent may be suitable for use as a DDAH1 agonist as described herein, e.g. in treating or preventing disorders characterised by endothelial dysfunction.

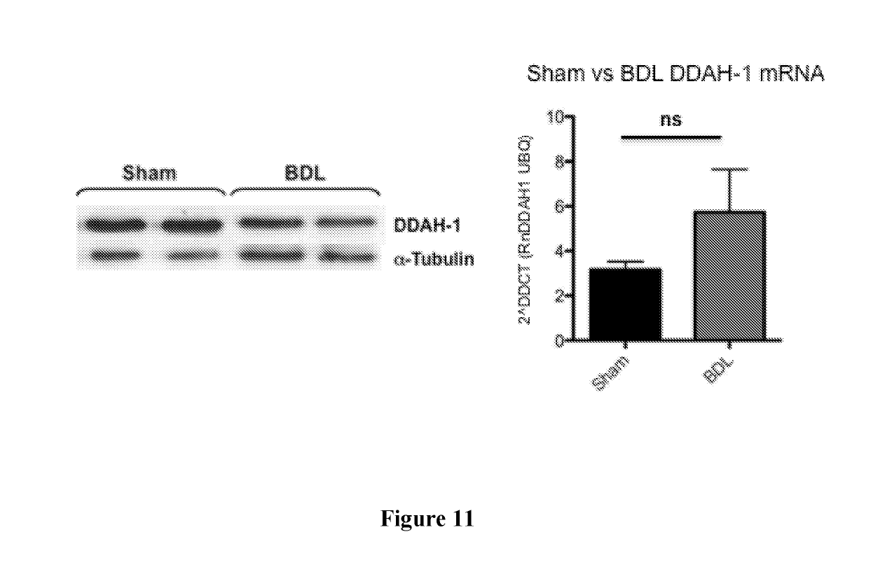

A screening method may therefore comprise contacting a test agent with one or more miRs of interest (such as any of those illustrated in FIG. 11) and determining whether the test agent is capable of binding to the miR(s). Such a method may further comprise altering the hybridisation conditions to determine the strength of binding of the test agent to the miR(s). A preferred agent will bind strongly and/or irreversibly to the miR(s) of interest.

A screening method may comprise contacting a test agent with one or more miR(s) of interest as described herein and further contacting said miR(s) with a DDAH1 mRNA under conditions under which expression of DDAH1 protein from said mRNA occurs in the absence of the test agent. The effect of the test agent on DDAH1 expression can then be assessed, e.g. by comparing the amount of DDAH1 protein that is produced in the presence of the test agent with the amount of DDAH1 protein that is produced in the absence of the test agent. An increase in the production of DDAH1 protein in the presence of the test agent indicates that test agent may be suitable for use as a DDAH1 agonist as described herein, e.g. in treating or preventing disorders characterised by endothelial dysfunction.

A test agent for use in a screening method of the invention refers to any compound, molecule or agent that may potentially block or reduce the effects of one or more miRs that bind in the 3′UTR of DDAH1, such as one or more of the miRs illustrated in FIG. 11. The test agent may be, or may comprise, for example, a peptide, polypeptide, protein, polynucleotide, oligonucleotide, small molecule or other compound that may be designed through rational drug design starting from known agonists of DDAH1.

The test agent to be screened could be derived or synthesised from chemical compositions or man-made compounds. Candidate agents may be obtained from a wide variety of sources including libraries of synthetic or natural compounds. Suitable test agents which can be tested in the above assays include compounds derived from combinatorial libraries, small molecule libraries and natural product libraries, such as display (e.g. phage display) libraries. Multiple test agents may be screened using a method of the invention in order to identify one or more agents having a suitable effect on DDAH1, such as stimulation of DDAH1 activity or expression.

The screening methods of the invention may be carried out in vivo, ex vivo or in vitro. In particular, the step of contacting a test agent with miR(s) and/or DDAH1 mRNA or with a cell or tissue that comprises such miR(s) and/or mRNA may be carried out in vivo, ex vivo or in vitro. The screening methods of the invention may be carried out in a cell-based or a cell-free system. For example, the screening method of the invention may comprise a step of contacting a cell or tissue comprising DDAH1 mRNA and miRs with a test agent and determining whether the presence of the test agent leads to an increase in the amount of DDAH1 protein in the cell or tissue.

A screening method of the invention may use a cell-free assay. For example, the miR(s) and/or mRNA may be present in a cell-free environment. A suitable cell-free assay may be carried out in a cell extract. For example, the contacting steps of the methods of the invention may be carried out in extracts obtained from cells that are capable of expressing DDAH1. A cell-free system comprising DDAH1 mRNA may be incubated with the other components of the methods of the invention such a test agent. In such a cell-free method, the amount of DDAH1 may be assessed in the presence or absence of a test agent in order to determine whether the agent is altering the amount of DDAH1 in the cell or tissue. The presence of an increased amount of DDAH1 in the presence of the test agent indicates that the test agent may be a suitable agonist of DDAH1 for use in accordance with the present invention.

Any suitable technique may be used to measure the enhancement or increase in the expression of DDAH1 or the binding of a target agent to miR(s). For instance, RNA solution hybridization, nuclease protection, Northern hybridization, reverse transcription, gene expression monitoring with a microarray, antibody binding, enzyme linked immunosorbent assay (ELISA), Western blotting, radioimmunoassay (MA), other immunoassays, and fluorescence activated cell analysis (FACS) may be employed.

### Pharmaceutical Formulations

A DDAH1 agonist as described herein may be provided in a pharmaceutical composition. It may thus be formulated for administration with a pharmaceutically acceptable carrier or diluent. The agonist may be any agonist as defined herein including any agonist identified by a screening method of the invention. The agonist may thus be formulated as a medicament with a standard pharmaceutically acceptable carrier(s) and/or excipient(s) as is routine in the pharmaceutical art. The exact nature of the formulation will depend upon several factors including the desired route of administration. Typically, the agonist may be formulated for oral, intravenous, intragastric, intravascular or intraperitoneal administration.

The pharmaceutical carrier or diluent may be, for example, an isotonic solution such as physiological saline. Solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with ornithine and at least one of phenylacetate and phenylbutyrate, a pharmaceutically acceptable carrier, e.g. sterile water, olive oil, ethyl oleate, glycols, e.g. propylene glycol, and if desired, a suitable amount of lidocaine hydrochloride.

Where the agonist to be administered is a nucleic acid molecule, for example where the agonist is in the form of an expression vector, certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules.

Sterile injectable solutions may be prepared, for instance, by incorporating the agonist in the required amount in an appropriate solvent with one or a combination of ingredients enumerated above, as required, followed by filtered sterilization. In the case of sterile powders for the preparation of sterile injectable solutions, preferred methods of preparation include vacuum drying and freeze-drying which yields a powder of the active ingredient plus any additional desired ingredient from a previously sterile-filtered solution thereof.

Pharmaceutical compositions comprising agonists of the invention encompass any pharmaceutically acceptable salts, esters, or salts of such esters of the agonist. In certain instances, a composition of the invention may include more than one agonist of the invention. Accordingly, for example, the disclosure is also drawn to pharmaceutically acceptable salts, prodrugs, pharmaceutically acceptable salts of such prodrugs, and other bioequivalents may also be employed. Suitable pharmaceutically acceptable salts include, but are not limited to, sodium and potassium salts. A prodrug may, for instance, include the incorporation of additional nucleosides at one or both ends of an oligomeric compound which are cleaved by endogenous nucleases within the body, to form an active oligonucleotide agonist.

The agonist may be formulated with carriers that protect the agonist against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid.

A pharmaceutical formulation in accordance with the present invention may further comprise one or more additional therapeutic agents. For example, the formulation may comprise one or more DDAH1 agonists as defined herein. The formulation may comprise one or more DDAH1 agonists as described herein and also one or more additional therapeutic agents. Preferably the additional therapeutic agent(s) are agents which will assist in the treatment or prophylaxis of the individual to be treated. For example, one or more agents that are effective at increasing the expression or activity of DDAH1 may be administered as part of a formulation as described herein.

The pharmaceutical compositions may be formulated in unit dosage forms. In some cases the compositions may be formulated in ampoules. The pharmaceutical compositions may be included in a container, pack, or dispenser together with instructions for administration. The invention also provides a kit comprising an oligonucleotide of the invention and optionally instructions for administration to a patient in need thereof, preferably such a kit has the agonist provided in the form of a pharmaceutical composition of the invention. The kit may also include means for administering the agonist or composition, for instance a syringe or other appropriate delivery device. The kit may comprise any of the means of delivery discussed herein. In one instance, the kit also comprises lipofectin and in particular the oligonucleotide formulated with lipofectin.

### Treatment

Augmentation of DDAH1 is a therapeutic goal since it is the primary route of metabolism of ADMA (asymmetric dimethylarginine), a factor that is generally elevated in conditions characterised by endothelial dysfunction.

Accordingly, the present invention provides methods for the treatment or prevention of diseases or disorders that are characterised by endothelial dysfunction, the method comprising administering to a subject in need thereof an agonist of DDAH1 as described herein. In particular, the disease or disorder may be characterised by increased ADMA levels.

The disease or disorder may be selected from coronary heart disease, peripheral vascular disease, chronic kidney disease, hypertension such as systemic hypertension, pulmonary hypertension, renovascular hypertension, portal hypertension or pregnancy induced hypertension/pre-eclampsia, raised inter-cranial pressure, stroke and chronic liver disease. The method may be used to reduce portal blood pressure, for example in a subject with portal hypertension.

The disease or disorder may be a cardiovascular disorder. For example, the disease or disorder may be or may comprise the symptom of hypertension such as renovascular or pulmonary hypertension. The disease or disorder may be a cerebrovascular disorder such as stroke. The disease or disorder may be cirrhosis with portal hypertension. The disease or disorder may be hepato-renal or porto-pulmonary dysfunction in cirrhosis. The disease or disorder may be pregnancy induced hypertension or pre-eclampsia. The disease or disorder may be diabetes mellitus induced end-organ injury.

In related aspects, an agonist of DDAH1 may be provided for use in a method of treating or preventing such a disease or disorder that is characterised by endothelial dysfunction. Also provided is the use of an agonist of DDAH1 in the manufacture of a medicament for treating or preventing a disease or disorder characterised by endothelial dysfunction. In any of these method or use embodiments, the agonist may be any agonist described herein and the disease or disorder may be any disease or disorder as described herein.

In any of these methods and uses, the DDAH1 agonist may be any DDAH1 agonist described herein, and in particular an agonist that acts to prevent the inhibition of DDAH1 expression by one or more miRs. The DDAH1 agonist may be any agonist identified by a screening method as described herein. The agonist may be provided in a formulation or composition as described herein.

An agonist of DDAH1 as described herein is thus administered to a subject in order to prevent or treat any of the conditions discussed above. An agonist of DDAH1 as described herein can thus be administered to improve the condition of a subject, for example a subject suffering from a disease or disorder characterised by endothelial dysfunction. An agonist of DDAH1 as described herein may be administered to alleviate the symptoms of a subject, for example the symptoms associated with a disease or disorder characterised by endothelial dysfunction. An agonist of DDAH1 as described herein may be administered to combat or delay the onset of a disease or disorder characterised by endothelial dysfunction or any symptom associated therewith. The invention can therefore prevent the medical consequences of a disease or disorder characterised by endothelial dysfunction. Use of an agonist of DDAH1 as described herein may thus extend the life of a patient with a disease or disorder characterised by endothelial dysfunction.

As described herein, the agonist of DDAH1 may lead to increased levels of DDAH1 in cells, an organ or tissue of the subject. The agonist may be targeted to the cells, tissue or organ of interest either through targeted administration, such as administration directly into the cells/tissue/organ, or targeted expression such as using a promoter that is specific to a particular cell, tissue or organ type. Single or multiple administrations of such an agonist to the individual may be used.

The subject is treated with an agonist of DDAH1 as described herein. As described above, the agonist of DDAH1 may be administered alone or in the form of a pharmaceutical formulation. The formulation may comprise one or more agonists of DDAH1 and may comprise one or more additional therapeutic or prophylactic agents.

Two or more different DDAH1 agonists as described herein may be used in combination to treat a subject. The two or more agonists may be administered together, in a single formulation, at the same time, in two or more separate formulations, or separately or sequentially as part of a combined administration regimen.

An agonist or formulation of the invention may be administered by any suitable route. Examples of routes of administration which may be employed in the invention, and which in some cases the composition may be formulated to aid compatibility with include parenteral, e.g., intravenous, intradermal, subcutaneous, intraperitoneal, intragastric, intramuscular, oral, inhalation, transdermal, topical, transmucosal, and direct delivery into an organ of interest may be employed. The agonists of the invention may be delivered by such routes.

In some embodiments it may be preferred to directly deliver the agonist to the organ of interest. This approach may be useful where there is a risk of side effects due to, for example, the agonists having possible effects in other cells or other tissue types. The agonist or formulation of the invention may therefore be administered directly to the organ, cell or tissue of interest. For example, if the treatment is intended to relate to a disorder of the liver, then the agonist or formulation of the invention may be delivered directly into the liver.

The agonist is administered in a therapeutically effective amount. A suitable dose of an agonist of the invention can be determined according to various parameters such as the age, weight and condition of the subject to be treated; the type and severity of the disease; the route of administration; and the required regimen. A suitable dose can be determined for an individual agonist. For example, for some agonists a typical dose may be in the order of from 1 mg/kg/day to 30 g/kg/day. Examples of intracellular concentrations of the agonist include those in the range from about 0.005 to 50 μM, or more preferably 0.02 to 5 μM. For administration to a subject such as a human, a daily dosage ranging from about 0.001 to 50 mg/kg, preferably 0.01 to 10 mg/kg, and more preferably from 0.1 to 5 mg/kg may be employed. The skilled person and particularly an appropriate physician will be able to identify an appropriate dosage, for instance taking factors such as age, sex, weight and so on into account.

The present invention is broadly applicable to therapeutic methods and is relevant to the development of prophylactic and/or therapeutic treatments. It is to be appreciated that all references herein to treatment include curative, palliative and prophylactic treatment.

Prophylaxis or therapy includes but is not limited to eliciting an effective increase in DDAH1 in order to cause a reduction in ADMA levels or an improvement in a disease or disorder characterised by endothelial dysfunction, such as to achieve a reduction in portal pressure, or in order to prevent or reduce an increase in portal pressure. For example, prophylaxis or therapy may result in the reduction of portal pressure in a subject with increased portal pressure such as a subject with portal hypertension. Prophylaxis or therapy may result in the maintenance of a particular level of portal pressure in a patient where portal pressure has been increasing or in which portal pressure is expected to increase. Prophylaxis or therapy may result in an increase in portal pressure in an individual being reduced or slowed compared to the increase that would have been seen, or would have been expected, in the absence of such treatment.

Prophylaxis or therapy may have similar effects in relation to any of the symptoms or consequences of conditions characterised by endothelial dysfunction described herein. That is, treatment in accordance with the present invention may lead to a lessening in the severity of such symptoms or consequences, maintenance of an existing level of such symptoms or consequences or a slowing or reduction in the worsening of such symptoms or consequences.

Where the agonist is a nucleic acid molecule, it may be introduced directly into the recipient subject, or can be introduced ex vivo into cells which have been removed from a subject. In this latter case, cells containing the nucleic acid may be re-introduced into the subject at a suitable location, such as to the liver of the subject. Various approaches for such gene delivery are known in the art and would be appreciated by the skilled reader. For example, a nucleic acid agonist could be delivered as a naked nucleic acid construct, preferably further comprising flanking sequences homologous to the host cell genome. The nucleic acid agonist could be delivered in a vector such as a plasmid vector, or a viral vector. Suitable recombinant viral vectors include but are not limited to adenovirus vectors and adeno-associated viral (AAV) vectors. For example, transduction of hepatocytes and other cell types in rodent models of liver disease has been reported using adenovirus vectors (Yu et al. Am J Phys 2002, 282: G565-G572, Garcia-Banuelos et al. Gene Therapy 2002, 9: 127-134). Such adenovirus vectors may be used in accordance with the present invention. Similarly, liver transduction of the AAV2 genome with an AAV8 capsid (AAV2/8) has also been reported (Osman et al. Atherosclerosis 2009, 204: 121-6). Such AAV2/8 vectors may also be used in accordance with the present invention. The nucleic acid could be administered in a liposomal preparation such as a cationic liposomal preparation.

In some embodiments, an agent that targets one or more of the miRs that repress DDAH1 protein translation from DDAH1 mRNA as described herein can be used in combination with a nucleic acid molecule encoding DDAH1 itself. The DDAH1 encoding nucleic acid molecule is administered to increase the level of expression of DDAH1 mRNA whilst inhibition of one or more miRNAs that regulate translation of DDAH1 serves to increase the extent to which that mRNA is translated.

In one embodiment the agonist comprising the DDAH1 nucleic acid molecule could be delivered in a vector, such as a plasmid vector, or a viral vector, for example an adenovirus vector or adeno-associated viral (AAV) vector.

Nucleic acids may be introduced into cells using any suitable method. For instance, transfection, electroporation, fusion, liposomes, colloidal polymeric particles and viral and non-viral vectors as well as other means known in the art may be used to deliver the oligonucleotide sequences to cells. In some instances, the oligonucleotide may be delivered using methods involving liposome-mediated uptake. Lipofectins and cytofectins are lipid-based positive ions that bind to negatively charged nucleic acid and form a complex that can ferry the nucleic acid across a cell membrane and may be employed. In one instance a lipofectin is used in the delivery of the oligonucleotide of the invention, particularly Lipofectamin2000.

Physical methods of introducing nucleic acids that comprise RNA include injection of a solution containing the RNA, bombardment by particles covered by the RNA, soaking the cell or organism in a solution of the RNA, or electroporation of cell membranes in the presence of the RNA. A viral construct packaged into a viral particle can be used to achieve efficient introduction into a cell and transcription of RNA encoded by the expression construct. Other methods known in the art for introducing nucleic acids to cells may be used, such as lipid-mediated carrier transport, chemical-mediated transport, such as calcium phosphate, and the like. RNA may be introduced along with components that perform one or more of the following activities: enhance RNA uptake by the cell, inhibit annealing of single strands, stabilize the single strands, or other-wise increase inhibition of the target gene.

The nucleic acids may be modified so that they target specific cells, for instance by binding to receptors found on a particular cell type. The nucleic acids reagents may be delivered to cells using a vector. The invention also provides a vector capable of expressing a nucleic acid DDAH1 agonist of the invention as well as a host cell comprising such an agonist.

Production of nucleic acid DDAH1 agonists may be targeted by specific transcription in an organ, tissue, or cell type; stimulation of an environmental condition (e.g., infection, stress, temperature, chemical inducers); and/or engineering transcription at a developmental stage or age. A transgenic organism that expresses an oligonucleotide reagent from a recombinant construct may be produced by introducing the construct into a zygote, an embryonic stem cell, or another multipotent cell derived from the appropriate organism. Liposomes may be used to achieve targeting by having specific markers on the surface for directing the liposome. Other means of targeting include injection directly into the tissue containing the target cells.

Cells targeted or used in the methods of the invention are preferably mammalian cells, in particular, human cells. Cells may be from the germ line or somatic, totipotent or pluripotent, dividing or non-dividing, parenchyma or epithelium, immortalized or transformed. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. Neurons and muscle cells (for instance myocytes, myoblasts, myotubes, myofibers, and the like) are preferred target cells.

The nucleic acid DDAH1 agonist reagent may be introduced in an amount which allows delivery of at least one copy per cell. Higher doses (for instance least 5, 10, 100, 500 or 1000 copies per cell) of material may yield greater effects. The amount administered will be enough to produce an observable improvement, for instance at the RNA, protein or level of symptoms displayed by a subject.

### Patients to be Treated

The present invention relates to the treatment or prevention of diseases or disorders characterised by endothelial dysfunction in individuals in need thereof. A subject to be treated in accordance with the present invention may therefore have a disease or disorder characterised by endothelial dysfunction or may be at increased risk of a disease or disorder characterised by endothelial dysfunction. For example, the subject may have coronary heart disease, peripheral vascular disease, chronic kidney disease, hypertension such as systemic hypertension, pulmonary hypertension, renovascular hypertension, portal hypertension or pregnancy induced hypertension/pre-eclampsia, raised inter-cranial pressure, stroke or chronic liver disease. Methods for diagnosing such conditions are well known in the art and in particular to clinicians and veterinarians in the field. Preferably, the subject will have been diagnosed as having such a disease or disorder, for example by a medical or veterinarian professional. The subject may display one or more symptoms associated with such a disease or disorder.

The subject to be treated may be a human. The subject to be treated may be a non-human animal such as a non-human mammal. The subject to be treated may be a farm animal for example, a cow or bull, sheep, pig, ox, goat or horse or may be a domestic animal such as a dog or cat. The subject may or may not be an animal model for liver disease. The animal may be any age, but will often be a mature adult subject.

TABLE 1
Mature miR sequences
for preferred miRs

| | Transcript | Sequence | |
|---|---|---|---|
| | (if | 5p sequence | 3p sequence |
| miR | applicable) | (if applicable) | (if applicable) |
| 30 | a | uguaaacauccuc | cuuucagucggau |
| | | gacuggaag | guuugcagc |
| | b | uguaaacauccua | cugggagguggau |
| | | cacucagcu | guuuacuuc |
| | c | uguaaacauccua | cugggagaggguu |
| | | cacucucagc | guuuacucc |
| | | | or |
| | | | cugggagaaggcu |
| | | | guuuacucu |
| | d | uguaaacaucccc | cuuucagucagau |
| | | gacuggaag | guuugcugc |
| | e | uguaaacauccuu | cuuucagucggau |
| | | gacuggaag | guuuacagc |
| 219 | a | ugauuguccaaac | agaguugagucug |
| | | gcaauucu | gacgucccg |
| | | | or |
| | | | agaauuguggcug |
| | | | gacaucugu |
| | b | agauguccagcca | agaauugcguuug |
| | | caauucucg | gacaaucagu |
| 508 | | uacuccagagggc | ugauuguagccuu |
| | | gucacucaug | uuggaguaga |
| 128 | | cggggccguagca | ucacagugaaccg |
| | | cugucugaga | gucucuuu |
| | | or | |
| | | gggggccgauaca | |
| | | cuguacgaga | |
| 21 | | uagcuuaucagac | caacaccagucga |
| | | ugauguuga | ugggcugu |
| 210 | | agccccugcccac | cugugcgugugac |
| | | cgcacacug | agcggcuga |
| 23 | a | gggguuccugggg | aucacauugccag |
| | | augggauuu | ggauuucc |
| | b | uggguuccuggca | aucacauugccag |
| | | ugcugauuu | ggauuacc |
| | c | aucacauugccag | |
| | | ugauuaccc | |
| 143 | | ggugcagugcugc | ugagaugaagcac |
| | | aucucuggu | uguagcuc |
| 4770 | | ugagaugacacug | |
| | | uagcu | |
| 96 | | uuuggcacuagca | aaucaugugcagu |
| | | cauuuuugcu | gccaauaug |
| 507 | | uuuugcaccuuuu | |
| | | ggagugaa | |
| 1271 | | cuuggcaccuagc | agugccugcuaug |
| | | aagcacuca | ugccaggca |
| 148 | a | aaaguucugagac | ucagugcacuaca |
| | | acuccgacu | gaacuuugu |
| | b | aaguucuguuaua | ucagugcaucaca |
| | | cacucaggc | gaacuuugu |
| 152 | | ucagugcaugaca | |
| | | gaacuugg | |
| 182 | | uuuggcaauggua | ugguucuagacuu |
| | | gaacucacacu | gccaacua |
| 27 | a | agggcuuagcugc | uucacaguggcua |
| | | uugugagca | aguuccgc |
| | b | agagcuuagcuga | uucacaguggcua |
| | | uuggugaac | aguucugc |
| 101 | | caguuaucacagu | uacaguacuguga |
| | | gcugaugcu | uaacugaa |
| 765 | | uggaggagaagga | |
| | | aggugaug | |
| 589 | | ugagaaccacguc | ucagaacaaaugc |
| | | ugcucugag | cgguucccaga |
| 1299 | | uucuggaauucug | |
| | | ugugaggga | |
| 595 | | gaagugugccgug | |
| | | gugugucu | |
| 301 | a | gcucugacuuuau | cagugcaauagua |
| | | ugcacuacu | uugucaaagc |
| | b | cagugcaaugaua | |
| | | uugucaaagc | |
| 548 | a | aaaaguaauugcg | caaaacuggcaau |
| | | aguuuuacc | uacuuuugc |
| | b | aaaaguaauugug | caagaaccucagu |
| | | guuuuggcc | ugcuuuugu |
| | c | aaaaguaauugcg | caaaaaucucaau |
| | | guuuuugcc | uacuuuugc |
| | d | aaaaguaauugug | caaaaaccacagu |
| | | guuuuugcc | uucuuuugc |
| | e | aaaaacugagacu | |
| | | acuuuugca | |
| | f | aaaaacuguaauu | |
| | | acuuuu | |
| | g | ugcaaaaguaauu | aaaacuguaauua |
| | | gcaguuuuug | cuuuuguac |
| | h | aaaaguaaucgcg | caaaaaccgcaau |
| | | guuuuuguc | uacuuuugca |
| | i | aaaaguaauugcg | |
| | | gauuuugcc | |
| | j | aaaaguaauugcg | |
| | | gucuuuggu | |
| | k | aaaaguacuugcg | |
| | | gauuuugcu | |
| | l | aaaaguauuugcg | |
| | | gguuuuguc | |
| | m | caaagguauuugu | |
| | | gguuuuug | |
| | n | caaaaguaauugu | |
| | | ggauuuugu | |
| | o | aaaaguaauugcg | ccaaaacugcagu |
| | | guuuuugcc | uacuuuugc |
| | p | uagcaaaaacugc | |
| | | aguuacuuu | |
| | q | gcuggugcaaaag | |
| | | uaauggcgg | |
| | s | auggccaaaacug | |
| | | caguuauuuu | |
| | t | caaaagugaucgu | aaaaaccacaauu |
| | | gguuuuug | acuuuugcacca |
| | u | caaagacugcaau | |
| | | uacuuuugcg | |
| | v | agcuacaguuacu | |
| | | uuugcacca | |
| | w | aaaaguaacugcg | |
| | | guuuuugccu | |
| | x | ugcaaaaguaauu | uaaaaacugcaau |
| | | gcaguuuuug | uacuuuc |
| | y | aaaaguaaucacu | |
| | | guuuuugcc | |
| | z | caaaaaccgcaau | |
| | | uacuuuugca | |
| | aa | aaaaaccacaauu | |
| | | acuuuugcacca | |
| | ab | aaaaguaauugug | |
| | | gauuuugcu | |
| | ac | caaaaaccggcaa | |
| | | uuacuuuug | |
| | ad | gaaaacgacaaug | |
| | | acuuuugca | |
| | ae | caaaaacugcaau | |
| | | uacuuuca | |
| | ag | aaagguaauugug | |
| | | guuucugc | |
| | ah | aaaagugauugca | caaaaacugcagu |
| | | guguuug | uacuuuugc |
| | ai | aaagguaauugca | |
| | | guuuuuccc | |
| | aj | ugcaaaaguaauu | uaaaaacugcaau |
| | | gcaguuuuug | uacuuuua |
| | ak | aaaaguaacugcg | |
| | | guuuuuga | |
| | al | aacggcaaugacu | |
| | | uuuguacca | |
| | am | aaaaguaauugcg | caaaaacugcagu |
| | | guuuuugcc | uacuuuugu |
| | an | aaaaggcauugug | |
| | | guuuuug | |
| | ao | agaaguaacuacg | aaagaccgugacu |
| | | guuuuugca | acuuuugca |
| | ap | aaaaguaauugcg | aaaaaccacaauu |
| | | gucuuu | acuuuu |
| | aq | gaaaguaauugcu | caaaaacugcaau |
| | | guuuuugcc | uacuuuugc |
| | ar | aaaaguaauugca | uaaaacugcaguu |
| | | guuuuugc | auuuuugc |
| | as | aaaaguaauugcg | uaaaacccacaau |
| | | gguuuugcc | uauguuugu |
| | at | aaaaguuauugcg | caaaaccgcagua |
| | | guuuuggcu | acuuuugu |
| | au | aaaaguaauugcg | uggcaguuacuuu |
| | | guuuuugc | ugcaccag |
| | av | aaaaguacuugcg | aaaacugcaguua |
| | | gauuu | cuuuugc |
| | aw | gugcaaaagucau | |
| | | cacgguu | |
| | ax | agaaguaauugcg | |
| | | guuuugcca | |
| | ay | aaaaguaauugug | caaaaccgcgauu |
| | | guuuuugc | acucuugca |
| | az | caaaagugauugu | aaaaacugcaauc |
| | | gguuuuugc | acuuuugc |
| 1261 | | auggauaaggcuu | |
| | | uggcuu | |
| 943 | | cugacuguugccg | |
| | | uccuccag | |
| 635 | | acuugggcacuga | |
| | | aacaaugucc | |
| 509 | | uacugcagacagu | ugauugguacguc |
| | | ggcaauca | uguggguag |
| | | or | |
| | | uacugcagacgug | |
| | | gcaaucaug | |
| 1231 | | gugucugggcgga | |
| | | cagcugc | |
| 653 | | guguugaaacaau | |
| | | cucuacug | |
| 1252 | | agaaggaaauuga | |
| | | auucauuua | |

### EXAMPLES

### Materials and Methods

### BDL Rats

These experiments utilised an established animal model of cirrhosis, the bile duct ligated (BDL) rat. BDL rats may be generated by methods known in the art. For example, male Sprague-Dawley rats (200-250 g) may be used for this procedure. Following anaesthetisation, a mid-line laparotomy may be performed, the bile duct exposed, triply ligated with 4.0 silk suture, and severed between the second and third ligature. The wound is then closed in layers with absorbable suture, and the animal allowed to recover in a quiet room before being returned to the animal storage facility.

### DDAH-Activity Assay

The following experimental conditions were used to determine DDAH activity.

Liver tissue samples (100 mg frozen tissue per 300 μL lysis buffer) were homogenized in Tris-HCl buffer in the presence of a protease inhibitor. The homogenate was centrifuged in a pre-cooled (4° C.) centrifuge for 90 min at 10,000 rpm. For the DDAH activity assay 50 μL aliquots of the resulting supernatant was added to 50 μL aliquots of PBS buffer containing 1 μL ¹⁴C L-NMMA (0.02 mCi) and 2 μL 100 mM unlabelled L-NMMA and incubated for 60 min at 37° C. After incubation, samples were prepared for determination of [¹⁴C] citrulline content by vortex-mixing with 1 ml of 50% (w/v) Dowex (pH 7.0) and centrifugation at 10,000 rpm for 5 min; 500 μL of the supernatant was then be mixed with 5 ml of liquid-scintillation fluid and assessed for scintillation counting on a liquid scintillation analyser (Packard Biosciences, Berks, UK.). One unit of the enzyme activity is defined as the amount that catalyzes formation of 1 μM L-citrulline from ADMA per min at 37° C.

### DDAH-qPCR Assay

Liver tissue samples were snap frozen in liquid nitrogen, and RNA was extracted using the RNeasy kit (Qiagen) according to manufacturer's instructions. RNA was reverse transcribed to cDNA using Superscript II reverse transcriptase according to manufacturer's instructions, and quantitative PCR (qPCR) was performed using Taqman (Life Technologies) FAM labeled probes to HsDDAH1 (transcripts NM_012137.3 and NM_001134445.1) and to the housekeeping control gene Hs peptidylprolyl isomerase A (PPIA) (transcript NM_021130.3). Quantitative PCR was performed using the Taqman Universal PCR Mastermix (Life Technologies) according to manufacturer's instructions. Samples were analysed in duplicate, and the mean Cₜ value was analysed. The results were analysed according to the comparative Cₜ method, or the 2^{-[delta][delta]Ct} method.

### Molecular Analysis: Western Blot

Extracts containing equal amounts of protein were denatured and separated on 4-12% NuPAGE Bis-Tris Gels and blotted on to PVDF membranes (Invitrogen, UK). The membranes were then being incubated with different goat anti DDAH1&2 antibodies (1:1000, respectively) and mouse anti-eNOS and iNOS (1:500&1:10,000 respectively; Transduction Laboratories/Pharmingen, San Jose, Calif.), rabbit anti-TNF-α (1:1000; abcam), rabbit anti-ADMA (1:1000; immundiagnostik) and mouse anti-CTH (1:1000; abnova) antibodies and later with respective HRP-conjugated secondary antibodies. The bands were visualized using an enhanced ECL detection kit and quantified by densitometry. Loading accuracy was evaluated via membrane rehybridization with antibodies against mouse and rabbit anti-α tubulin (1:1000; upstate and Cell Signaling Technology, respectively).

### ADMA, SDMA and Arginine Measurement:

ADMA, SDMA and arginine were measured using fragmentation specific stable isotope dilution electrospray tandem mass spectrometry. In brief, samples de-proteinized with deuterated ADMA, SDMA and arginine, were chromatographed (acetonitrile:water, 1:1, with 0.025% formic acid) on a Teicoplanin guard column 10 mm×2.1 mm ID (Chirobiotic T, ASTEC Ltd, Congleton, UK), and analysed using a SCIEX API4000 (Applied Biosystems, Warrington, UK) in positive ion multiple reaction monitoring mode.

### Bioinformatics

Several online miRNA binding site prediction programmes were used to predict miRNA binding sites in the DDAH1 3′UTR. The following versions were used: Targetscan Human v5.2, Targetscan Human v6.2, miRBase v19, Pictar (2007), miRanda 4.0.

miRNA Luciferase Reporter Assays

The full-length 3′UTR of DDAH1 mRNA (sequence as in FIG. 17) was subcloned into the pMIRReport luciferase reported vector (Ambion). Subsequently, shorter sub-sections of the 3′UTR were subcloned into the pMIRReport vector for further identification of relevant regulatory areas. Luciferase assays were performed 24 hours after co-transfection of the pMIRReport vector and a pCMV_Renilla vector into HepG2 cells by standard techniques, using a Dual Luciferase Reporter Assay system (Promega).

Transfection of miRNA Mimics miRNA mimics (Qiagen) were transfected into HepG2 cells using the Hiperfect (Qiagen) transfection reagent according to manufacturer's instructions. Western blot was performed to assess DDAH1 protein expression 24 hours following transfection of miRNA mimics into the HEK293T cell line.
2-Plex In Situ Hybridisation of mRNA and miR

Bile-duct ligated (BDL) rats (as a model of liver disease) were sacrificed 4 weeks post-surgery. Sham rats were used as control. Portal pressure was measured as described in examples above. A vertical portion of liver lobe was removed from each animal and fixed in 10% neutral-buffered formalin (NBF) for 24 hours, and then processed and paraffin-embedded. 3-5 μm sections were used in 2-plex in situ hybridization (ISH) to detect DDAH1 mRNA and miR-128.

### Example 1

### Infliximab Treatment Increases DDAH1 Levels and Reduces Portal Pressure

Three groups of rats were used in these experiments, BDL rats treated with vehicle, BDL rats treated with the anti-TNF monoclonal antibody infliximab, and sham treated rats.

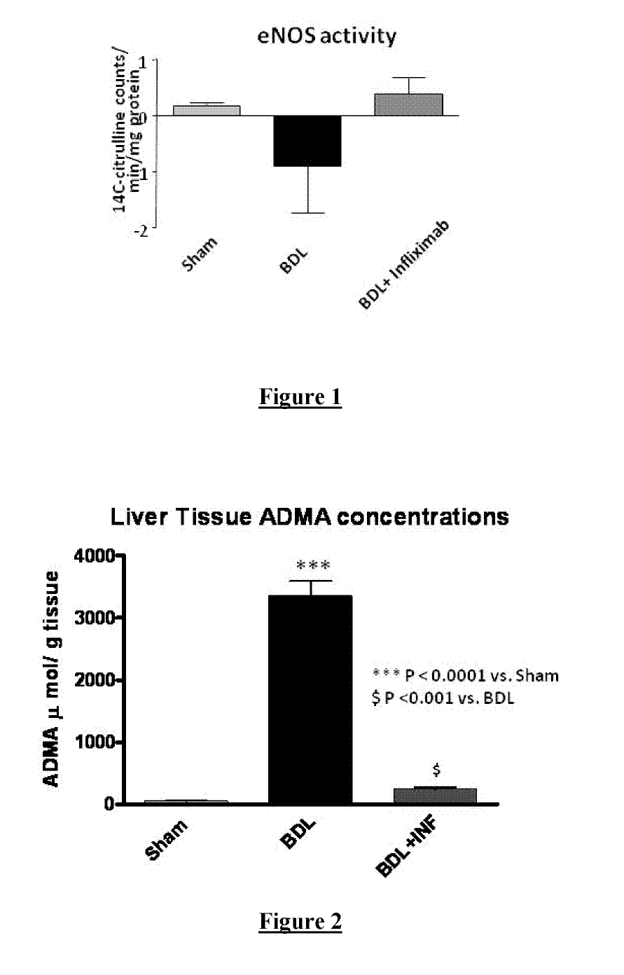

As shown in FIG. 1, bile duct ligation was found to markedly reduce eNOS activity, but treatment of BDL rats with infliximab restored eNOS activity towards similar levels to those seen in the sham animals.

As shown in FIG. 2, bile duct ligation also led to a significantly elevated liver tissue ADMA concentration compared with sham animals. However ADMA levels were substantially reduced by treatment with infliximab.

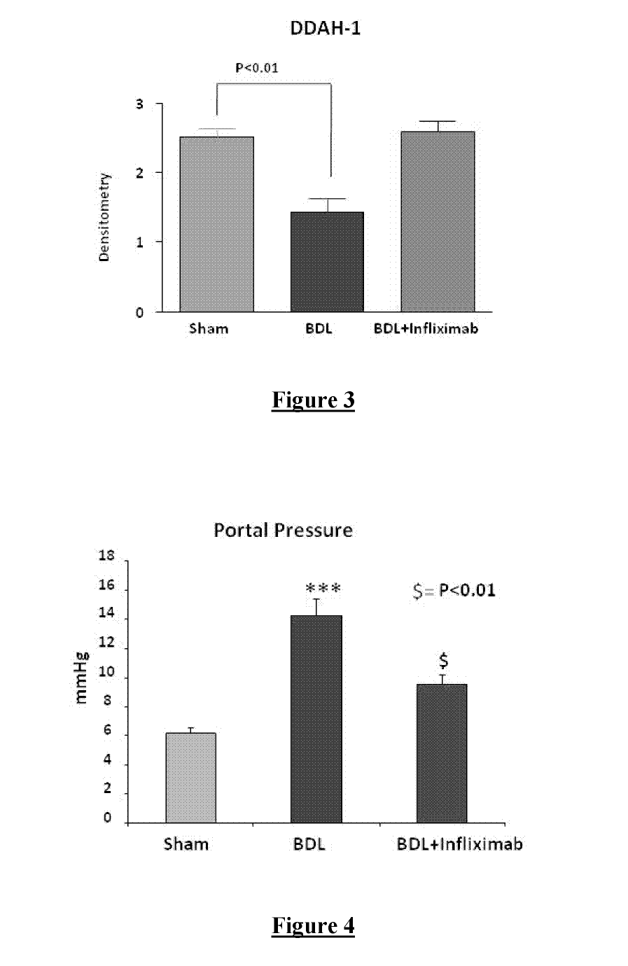

As shown in FIG. 3, bile duct ligation markedly reduced expression of the DDAH1 isoform in the livers. Upon treatment with infliximab, the DDAH1 expression levels were restored towards sham levels.

As shown in FIG. 4, portal pressure was markedly increased in BDL cirrhotic rats compared to normal sham portal pressures (14±0.7 vs. 5.5±0.3 mmHg). Following intervention with Infliximab, this was reduced by more than 30% (9.5±0.6 mmHg).

The Inventors have thus found that treatment of BDL rats with infliximab led to
A significant increase in hepatic tissue DDAH 1 expression.
A reduction in hepatic ADMA generation.
Increased hepatic NO generation (NOS activation).
A greater than 33% reduction in portal pressure compared to BDL rats treated with iso-volumetric saline injection.

### Example 2

### The Farnesoid Receptor Agonist INT-747 Increases DDAH1 Levels and Reduces Portal Pressure

The Farnesoid receptor (FXR) is a bile-acid responsive nuclear receptor previously shown to have hepatoprotective effects from bile duct ligation (BDL) injury in rats. FXR agonists have numerous target genes including DDAH1.

Four weeks after BDL or sham surgery in Sprague-Dawley rats (n=14), BDL rats were gavaged with 5 mg/kg of the FXR agonist INT-747 (obeticholic acid, Intercept Pharmaceuticals Inc.) in vehicle (corn oil) for 5 days or with vehicle alone.

After 5 days of treatment rats underwent direct portal pressure assessment and were then sacrificed, and plasma and liver tissue collected for analysis. eNOS activity was determined radiometrically by the conversion of labelled radioactive arginine to citrulline. Protein expression for eNOS, iNOS, DDAH1, and DDAH2 were measured by standard Western Blotting techniques. Liver biopsies were evaluated for histopathology with H+E, Van Gieson and reticulin stains.

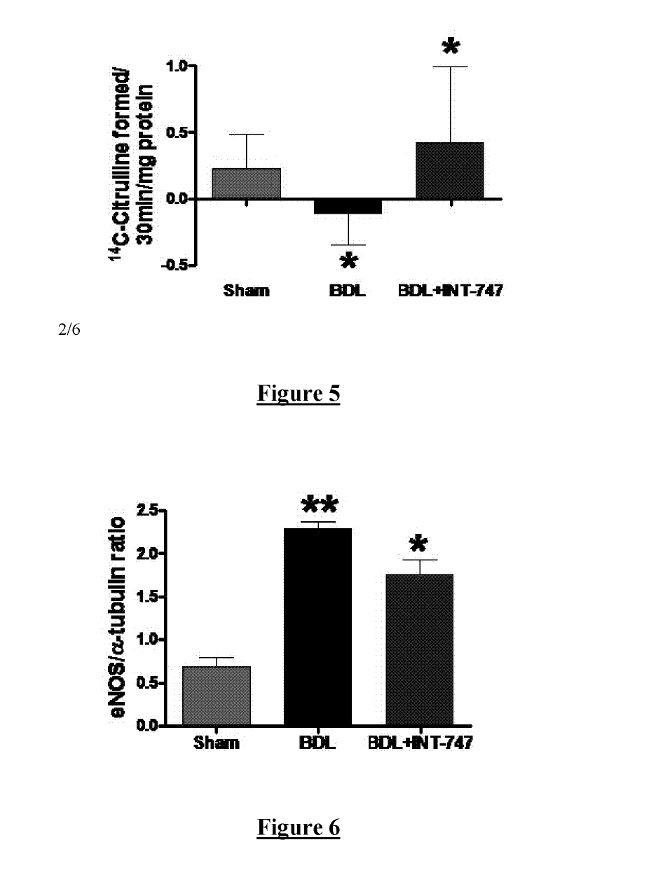

As shown in FIGS. 5 and 6, following treatment with INT-747, eNOS activity in BDL rats reverted to sham levels (*-p<0.05), with similar normalisation of eNOS protein expression (*-p<0.05)

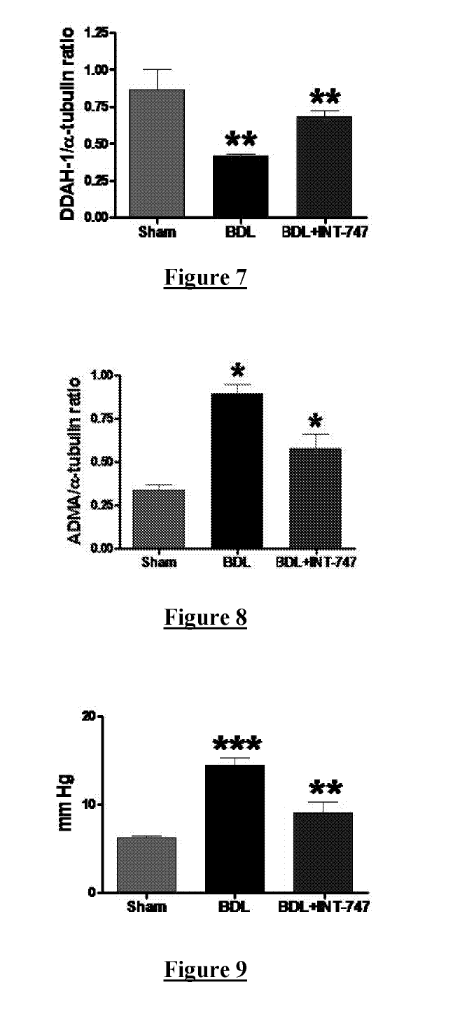

As shown in FIG. 7, INT-747 administration to BDL rats led to a significant increase in DDAH1 expression (**-p<0.01) with a significant reduction in ADMA (*-p<0.05) compared with BDL alone.

As shown in FIG. 9, INT-747 treatment in BDL rats led to a 30% reduction in portal pressure when compared to BDL+vehicle (**-p<0.01). This reduction in portal pressure following intervention with INT-747 occurred in the absence of any significant change in histological fibrosis or inflammation

Plasma TNFα levels were not significantly altered in the animals treated with INT-747 when compared with the BDL+vehicle animals.

### Example 3

The injection of naked plasmid DNA with a promoter that is efficient in mammalian cells has been demonstrated to result in effective liver transduction of the gene of interest in rodents (Maruyama et al. J Gene Med 2002, 4: 333-41). This method, termed ‘hydrodynamic gene therapy’, was used to determine the effect of transduction of DDAH1 in BDL rats on portal pressure.

A plasmid containing human DDAH1 cDNA was injected via a branch of the jugular vein into BDL or sham rodents produced as above. A control, non-expressing, plasmid was used as a control for the intervention in BDL animals. The animals underwent direct portal pressure measurement at 72 hours post intervention and plasma and tissue were collected for analysis of liver DDAH1 mRNA (qPCR) and protein expression (western blotting), as well as routine histology and biochemistry.

All the treated animals tolerated the hydrodynamic injection well and were given access to chow and water ad libitum. After 72 hours, at the time of sacrifice, portal pressure assessments were made.

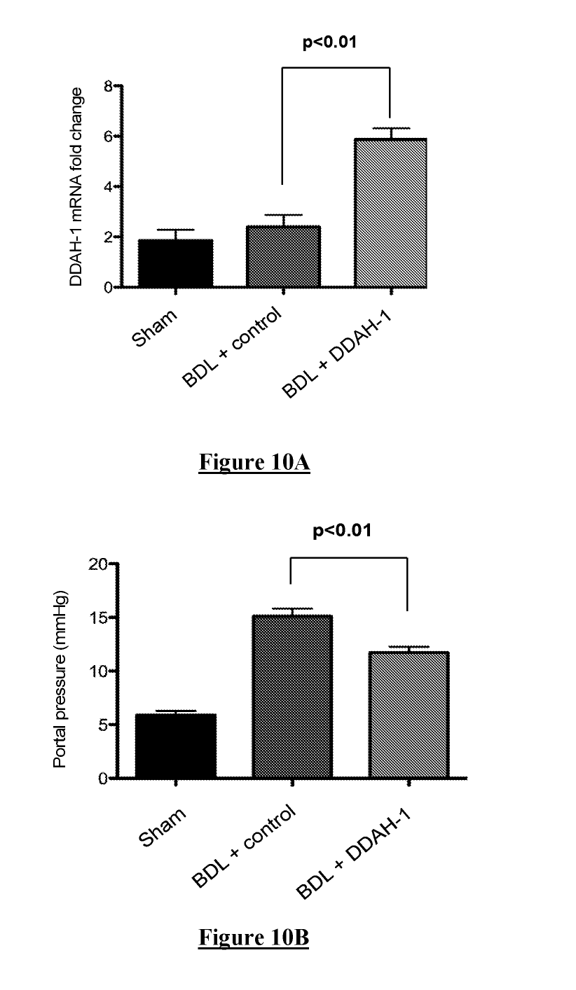

Sham rats had a mean portal pressure that was significantly lower than BDL rats injected with control plasmid (FIG. 10A). However, the group that had been treated with the DDAH1 incorporated plasmid had a significant increase in DDAH1 mRNA (FIG. 10A) and protein expression (data not shown) and a significant decrease in portal pressure compared with the control plasmid treated group (FIG. 10B). This data confirms the assertion that genetic therapy by reconstitution of DDAH1 cDNA may act as a therapy for the treatment of portal hypertension.

### Example 4

Adeno-associated virus (AAV) vectors are amongst the most frequently used viral vectors for gene therapy. Efficient liver transduction of the AAV2 genome with an AAV8 capsid (AAV2/8) has been demonstrated, when injected intravenously into mice (Osman et al; Atherosclerosis 2009, 204: 121-6). Expression of the gene of interest was driven by a truncated liver-specific promoter, LP1, containing segments of the human apoE/CI hepatic control region (HCR) and alpha-1-antitrypsin (hAAT) gene promoter, providing strong hepatic-restricted transgene production.

We have cloned a DNA construct containing human DDAH1 cDNA in the AAV2/8 plasmid, under the control of the liver-specific LP1 promoter.

The subsequent steps in creating the AAV vector are: (a) transfection of the DDAH-AAV2/8 plasmid and helper and packaging plasmids into competent cells, (b) purification of the DDAH-AAV2/8 with AVB Sepharose Column, and (c) quantification of DDAH-AAV2/8 with rtPCR.

The DDAH-AAV2/8 vector is injected via tail vein injection into BDL or sham rats. A negative control AAV2/8, without the gene of interest, is also injected into BDL rats. Portal pressure may be assessed by direct cannulation. Transduction of DDAH1 may be assessed by measurement of mRNA (rtPCR) and protein (western blotting).

### Example 5

Adenovirus vectors have been shown to demonstrate efficient transduction of hepatocytes and other cell types in rodent models of liver disease (Yu et al, Am J Phys 2002, 282: G565-G572; Garcia-Banuelos et at Gene Therapy 2002, 9: 127-134).

An adenovirus expressing DDAH1 is constructed. Once constructed the DDAH-adenovirus construct may be used to determine the effect of transduction of DDAH1 into hepatocytes and non-parenchymal cells (including sinusoidal endothelial cells) on eNOS activity portal pressure in BDL rats.

The DDAH-adenovirus vector is injected via tail vein injection into BDL sham rats. A negative control AAV2/8, without the gene of interest, is also injected into BDL rats. Portal pressure may be assessed at 5 days by direct cannulation of the portal vein. Transduction of DDAH1 may be assessed by measurement of mRNA (rtPCR) and protein (western blotting).

### Example 6

### DDAH1 Protein Levels are Decreased in Disease, but mRNA Levels are Unchanged, Suggesting Post-Transcriptional Regulation

Bile-duct ligated (BDL) rats demonstrate cirrhosis with features of acute-on-chronic liver failure, including portal hypertension, acute kidney injury, brain swelling and hepatopulmonary syndrome. In cirrhotic BDL rats, DDAH1 protein levels are reduced (Western blot, FIG. 11 left panel), but DDAH1 mRNA levels are preserved (quantitative Taqman PCR, FIG. 11 right panel).

### Example 7

### Bioinformatic Software Predicts Several miR Binding Sites on the Human DDAH1 3′UTR

Three independent bioinformatic prediction software programmes (Targetscan, Pictar, miRanda) were used to predict miRNA binding sites on the 3′ untranslated region (3′UTR) of DDAH1 mRNA (FIG. 12):

As shown in FIGS. 12A and 12C, the 3′UTR region contains a number of predicted miRNA binding sites. As shown in FIG. 12B, FIG. 12D and FIG. 14 (discussed further below), the predicted binding sites for miR-219, miR-128 and miR-30 are found within a 52 bp region.

The 52 bp region and the predicted binding sites for these miRs are as follows:

```
g t t t t c c t t g a c a a t c t a c t g t g c
                                          miR-219
c a c t g t g c t a c t a a c t c t t g t t t a
    miR-128 miR-30
c a a a
```

### Example 8

### Candidate miRNAs are Elevated in Liver from Cirrhotic Rats

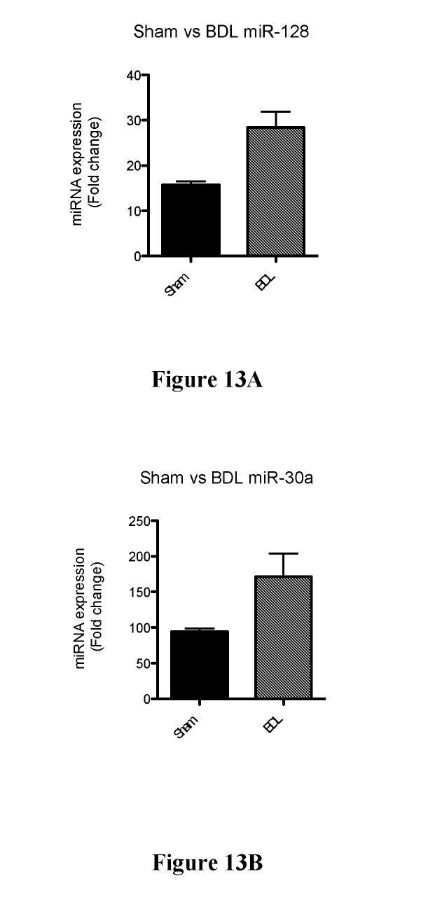

Hepatic miRNA expression was measured in a subset of 6 rats (sham n=3, BDL n=3) using the Affymetrix Genechip miRNA microarray. Predicted miRNAs from the bioinformatic analysis (eg. miR-128, miR-30a) are significantly elevated in BDL liver (FIG. 13).

### Example 9

### Luciferase Reporter Studies Demonstrate that the Predicted miRNA Sites in the DDAH1 3′UTR are Regulatory in Human Cell Lines

Luciferase reporter constructs were constructed to characterise the regulatory elements in the DDAH1 3′UTR in vitro (FIG. 15).

The 52 bp region including the predicted binding sites for miR-219, miR-128 and miR-30 (see Example 7, FIG. 14) was subcloned into the pMirReport luciferase vector and the vector was transfected into HepG2 cells. A number of separate vectors were generated, as illustrated in the right hand panel of FIG. 15. These includes a vector containing the full DDAH1 3′UTR sequence, a vector containing the 52 bp sequence, and a vector containing the 52 bp sequence into which a mutation had been introduced in the predicted binding site for miR 128.

As shown in FIG. 15, the presence of the full 3′UTR sequence repressed expression of the luciferase gene from the vector when compared to a control vector. The same effect was seen when only the 52 bp sequence was used, indicating that the 52 bp sequence is highly regulatory to the same degree as the entire DDAH1 3′UTR. The introduction of a mutation in the predicted miR-128 binding site region of the 52 bp sequence caused the loss of this repression. These data demonstrate that the entire DDAH1 3′UTR is regulatory in human cell lines, as is a 52 bp region containing the predicted binding sites for miRs 219-5p, 128, 30a.

### Example 10

### Candidate miRNAs Decrease Endogenous DDAH1 Expression in Human Cell Lines

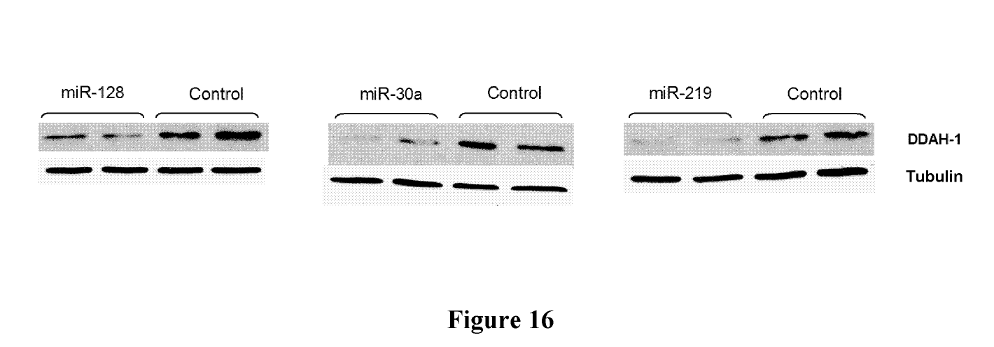

Transfection of miRNA analogues into for miRs 128, 219 and 30a in HEK293T cells leads to knockdown of endogenous DDAH1 protein expression compared with transfection of scrambled control (FIG. 16).

### Example 11

### Relative Expression of DDAH1 mRNA in Sham and BDL (Cirrhosis) Rat Liver

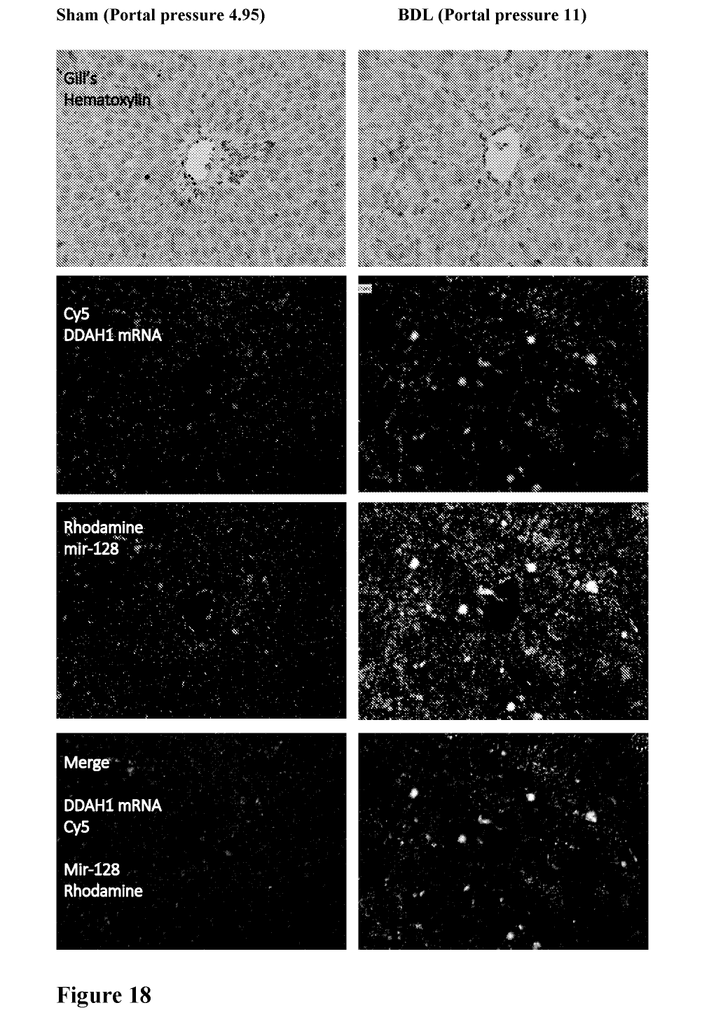

In-situ hybridisation was carried out using probes against DDAH1 mRNA and miR-128 (FIG. 18). BDL rats had significantly higher portal pressure than sham rats, confirming the development of cirrhosis and portal hypertension. The level of DDAH1 mRNA was increased (non-significantly) between BDL and sham rats despite previous data demonstrating lower DDAH1 protein expression, suggesting post transcriptional regulation. The level of miR-128 is markedly elevated in BDL rats compared to sham animals. This is consistent with the authors' previous data obtained by qPCR and western blot. The 2-plex ISH data suggests a co-localization of miR-128 and DDAH1 distributed widely throughout the cirrhotic liver specimens as shown in the merged figure.

### Example 12

### In Vivo miR-128 Knockdown

An in vivo proof of concept experiment is as follows. BDL rats are treated 2 weeks post surgery with miR-128 antisense sequences delivered via either locked nucleic acid (LNA) anti-sense oligonucleotides (ASOs) or as adeno-associated virus (AAV) particles expressing short hairpin sequences. Rats are sacrificed 2 weeks post treatment (4 weeks post BDL surgery). Placebo treated BDL rats and sham-operated are used as controls. Portal pressures are measured and tissues collected. The effect of miR-128 anti-sense treatment on portal pressure, DDAH1 protein expression, ADMA and NO generation is assessed.

1. A method of treating or preventing a disease or disorder characterised by endothelial dysfunction comprising administering to a subject in need thereof an agonist of DDAH1, wherein said agonist prevents, inhibits or reduces the microRNA mediated repression of DDAH1 protein translation from DDAH1 mRNA.

2. A method according to claim 1 wherein said agonist leads to:
(a) increased expression of DDAH1 protein in the subject; and/or
(b) increased levels of DDAH1 in the subject.

3. A method according to claim 2 wherein said agonist acts on DDAH1 in preference to DDAH2.

4. A method according to any one of the preceding claims wherein said microRNA is miR-128 and miR-219 or both.

5. A method according to claim 4 wherein miR-219 is 219-5p.

6. A method according to any one of the preceding claims wherein the agonist acts to prevent the effects of one or more miRs that repress DDAH1 protein translation from DDAH1 mRNA.

7. A method according to claim 5 wherein the miRs comprise:
(a) miR-128 and/or miR-219; and
(b) one or more other miRs selected from miR-30, miR-508, miR-23, miR-143, miR-1721, miR-4770, miR-96, miR-507, miR-1271, miR-148, miR-152, miR-182, miR-27, miR-101, miR-765, miR-589, miR-1299, miR-595, miR-301, miR-548, miR-1261, miR-943, miR-635, miR-509, miR-548, miR-1231, miR-653 and miR-1252; or 2, 3, up to 5, up to 10 or more, or all of these.

8. A method according to any one of the preceding claims wherein said agonist binds to said microRNA.
A method according to claim 8 wherein said agonist is a nucleic acid molecule comprising a sequence that is complementary to at least a part of said microRNA.

10. A method according to claim 8 wherein said agonist is a nucleic acid molecule that hybridises to said microRNA.

11. A method according to claim 8 wherein said agonist binds to said microRNA and thereby prevents the microRNA from interacting with the DDAH1 mRNA.

12. A method according to claim 9, 10 or 11 wherein said agonist is a locked nucleic acid (LNA).

13. A method according to claim 9, 10 or 11 wherein said nucleic acid molecule is expressed from an adenovirus vector or adeno-associated viral (AAV) vector.

14. A method according to any one of the preceding claims wherein said agonist is administered in combination with a nucleic acid molecule encoding DDAH1.

15. A method according to claim 14 wherein the DDAH1-encoding nucleic acid molecule is delivered in an adenovirus vector or adeno-associated viral (AAV) vector.

18. A method according to any one of the preceding claims wherein said disease or disorder is characterised by increased levels of asymmetric dimethylarginine (ADMA).

19. A method according to any one of the preceding claims wherein said subject has coronary heart disease, peripheral vascular disease, chronic kidney disease, hypertension such as systemic hypertension, pulmonary hypertension, renovascular hypertension, portal hypertension or pregnancy induced hypertension/pre-eclampsia, raised inter-cranial pressure, stroke or chronic liver disease.

20. An agonist of DDAH1 for use in a method of treating or preventing a disease or disorder characterised by endothelial dysfunction, wherein said agonist prevents, inhibits or reduces the microRNA mediated repression of DDAH1 protein translation from DDAH1 mRNA.

21. Use of an agonist of DDAH1 in the manufacture of a medicament for treating or preventing a disease or disorder characterised by endothelial dysfunction, wherein said agonist prevents, inhibits or reduces the microRNA mediated repression of DDAH1 protein translation from DDAH1 mRNA.

22. A method of identifying an agent suitable for use in treating portal hypertension, the method comprising determining whether a test agent is capable of binding to a microRNA selected from miR-128, miR-219 and miR-30, or a combination of any two or all three thereof.

23. A method according to claim 22, further comprising a step of contacting a cell or tissue comprising DDAH1 mRNA and said microRNA with said test agent and determining whether the presence of the test agent leads to an increase in the amount of DDAH1 protein that is produced in the cell or tissue.
